(19) United States
(12) Patent Application Publication
(10) Pub. No.: US 2022/0364160 A1
(43) Pub. Date: Nov. 17, 2022

(54) Amplification of RNA Detection Signals in Biological Samples

(71) Applicant: Akoya Biosciences, Inc., Menlo Park, CA (US)

(72) Inventors: Garry Nolan, Redwood City, CA (US); Julia Kennedy-Darling, Redwood City, CA (US); Peter J. Miller, Cambridge, MA (US)

(21) Appl. No.: 17/743,799

(22) Filed: May 13, 2022

Related U.S. Application Data
(60) Provisional application No. 63/189,056, filed on May 14, 2021.

(51) Int. Cl. C12Q 1/6818

(52) U.S. Cl. CPC C12Q 1/6818

(57) ABSTRACT

Methods include contacting a biological sample with a first probe, where the first probe includes a capture moiety having an oligonucleotide sequence that selectively binds to a RNA in the sample, and a secondary oligonucleotide region that does not bind to the RNA, contacting the sample with a second probe, where the second probe includes a probe binding region that is complementary to, and hybridizes to, a portion of the secondary oligonucleotide region, and includes a reporter moiety, and extending the secondary oligonucleotide region using the second probe as a template to generate an extended secondary oligonucleotide region featuring multiple copies of the reporter moiety, where the reporter moiety includes a plurality of label regions each featuring an oligonucleotide sequence, and one or more of the label regions of the reporter moiety are different from the other label regions of the reporter moiety.

### CROSS-REFERENCE TO RELATED APPLICATIONS

This application claims priority to U.S. Provisional Patent Application No. 63/189,056, the entire contents of which are incorporated herein by reference.

### TECHNICAL FIELD

This disclosure relates to labeling RNA in biological samples and detection of signals corresponding to the labeled RNA.

### BACKGROUND

Fluorescence imaging can be used to detect and quantify a wide variety of different analytes in biological samples. By labeling each different type of analyte with a different fluorescent dye, multiplexed detection of multiple analytes can be performed. In some assays, the number of different analytes that can be detected is limited by the number of distinct fluorescence measurement signals that can be resolved.

### SUMMARY

This disclosure features methods, compositions, and kits for labeling RNA in biological samples, measuring signals corresponding to the labeled RNA, and spatially localizing and quantifying the RNA in the sample. The methods include hybridizing a probe that specifically binds to the RNA, and then extending the probe such that the extended probe includes multiple different label regions. Multiple cycles of amplification can be performed to incorporate multiple copies of each of the different label regions in the extended probe. Each of the label regions has an oligonucleotide sequence, and the combination of label regions in the extended probe forms a reporter code that is specific to the extended probe. Hybridizing optical labels to each of the label regions and detecting signals generated by the optical labels allows the reporter code to be detected. Because the reporter code is uniquely associated with the RNA to which the probe binds, detection of the signals permits spatially localized detection of the RNA.

In an aspect, the disclosure features methods that include: contacting a biological sample with a first probe, where the first probe includes a capture moiety having an oligonucleotide sequence that selectively binds to a RNA in the sample, and a secondary oligonucleotide region that does not bind to the RNA; contacting the sample with a second probe, where the second probe includes a probe binding region that is complementary to, and hybridizes to, a portion of the secondary oligonucleotide region, and features a reporter moiety; and extending the secondary oligonucleotide region using the second probe as a template to generate an extended secondary oligonucleotide region including multiple copies of the reporter moiety, where the reporter moiety includes a plurality of label regions each having an oligonucleotide sequence, and where one or more of the label regions of the reporter moiety are different from the other label regions of the reporter moiety.

Embodiments of the methods can include any one or more of the following features.

The probe binding region can be a first probe binding region that is complementary to, and hybridizes to, a first portion of the secondary oligonucleotide region. The second probe can include a second probe binding region that is complementary to, and hybridizes to, a second portion of the secondary oligonucleotide region different from the first portion of the secondary oligonucleotide region. The methods can include, prior to extending the secondary oligonucleotide region, joining the first and second probe binding regions. The methods can include performing a ligation reaction to join the first and second probe binding regions.

The second probe can include a circular nucleic acid. Extending the secondary oligonucleotide region can include performing a rolling circle amplification reaction to extend the secondary oligonucleotide region. The extended secondary oligonucleotide region can include at least 10 copies (e.g., at least 50 copies, at least 100 copies) of the reporter moiety.

The reporter moiety can include at least 3 label regions (e.g., at least 4 label regions). One of the label regions of the reporter moiety can be different from the other label regions of the reporter moiety. Two of the label regions of the reporter moiety can be different from the other label regions of the reporter moiety. The reporter moiety can include at least two different types of label regions, where each different type of label region has a unique oligonucleotide sequence. The reporter moiety can include at least three different types (e.g., at least four different types) of label regions. Each of the label regions in the reporter moiety can be a different type of label region. Each label region can include at least 15 nucleotides (e.g., at least 30 nucleotides). Each label region can include a same number of nucleotides.

The methods can include: (a) exposing the sample to a plurality of optical labels, where each of the optical labels includes an oligonucleotide having a sequence, and a species that generates an optical signal; (b) measuring optical signals generated by optical labels of the plurality of optical labels that hybridize to complementary label regions of the reporter moieties; (c) repeating steps (a) and (b) with different pluralities of optical labels; (d) identifying one or more of the reporter moieties in the sample based on the measured optical signals; and (e) determining one or more locations of the RNA in the sample based on the one or more identified reporter moieties. The species that generates the optical signal can be a fluorescent moiety. The species that generates the optical signal can include at least one fluorescent nucleotide.

Measuring optical signals generated by the optical labels can include obtaining at least one image of the optical labels in the sample, and identifying optical signals corresponding to the optical labels in the at least one image. Each plurality of optical labels in step (a) can include a same number of different types of optical labels. Each plurality of optical labels in step (a) can include 3 or more different types of optical labels (e.g., 5 or more different types of optical labels). The methods can include repeating step (a) until the sample has been exposed to a set of optical labels, where each label region of the reporter moieties has a complementary optical label in the set of optical labels.

The methods can include exposing the sample to at least one of the plurality of optical labels more than once. Each time step (a) is performed, each member of the plurality of optical labels in step (a) can include a species that generates a different optical signal. The different optical signals can have different spectral distributions.

Among the plurality of optical labels, at least two of the optical labels can include a common species that generates the optical signal, and the at least two of the optical labels can be exposed to the sample during different repetitions of step (a). Among the plurality of optical labels, first and second optical labels each include a first species that generates the optical signals of the first and second optical labels, third and fourth optical labels each include a second species that generates the optical signals of the third and fourth optical labels, and the first and second species are different. The optical signals generated by the first and second species can be different. The methods can include exposing the sample to the first and second optical labels during different repetitions of step (a), and exposing the sample to the third and fourth optical labels during different repetitions of step (a).

The methods can include, for at least one of the optical labels, removing the at least one of the optical labels from the sample after measuring an optical signal generated by the at least one of the optical labels. Removing the at least one of the optical labels can include dehybridizing the at least one of the optical labels from one or more label regions of the reporter moieties.

Embodiments of the methods can also include any of the other features described herein, and can include any combination of features that are described in connection with different embodiments, except as expressly stated otherwise.

In another aspect, the disclosure features methods that include: contacting a biological sample with a first probe and a second probe, where the first probe includes a first capture moiety having an oligonucleotide sequence that selectively binds to a first portion of a RNA in the sample and a secondary oligonucleotide region that does not bind to the RNA, and where the second probe includes a second capture moiety having an oligonucleotide sequence that selectively binds to a second portion of the RNA and a secondary oligonucleotide region that does not bind to the RNA; contacting the sample with a third probe that includes a first probe binding region that is complementary to, and hybridizes to, a portion of the secondary oligonucleotide region of the first probe, and a second probe binding region that is complementary to, and hybridizes to, a portion of the secondary oligonucleotide region of the second probe; contacting the sample with a fourth probe comprising a third probe binding region that is complementary to, and hybridizes to, a portion of the secondary oligonucleotide region of the first probe, a fourth probe binding region that is complementary to, and hybridizes to, a portion of the secondary oligonucleotide region of the second probe, and a reporter moiety featuring a plurality of label regions each having an oligonucleotide sequence; and extending the secondary oligonucleotide region of the first probe using the fourth probe as a template to generate an extended secondary oligonucleotide region that includes multiple copies of the reporter moiety, where one or more of the label regions of the reporter moiety are different from the other label regions of the reporter moiety.

Embodiments of the methods can include one or more of the following features.

The methods can include, prior to extending the secondary oligonucleotide region of the first probe, joining the third and fourth probes to form a circularized probe featuring the reporter moiety. The methods can include performing a ligation reaction to join the third and fourth probes.

Extending the secondary oligonucleotide region of the first probe can include performing a rolling circle amplification reaction to extend the secondary oligonucleotide region. The extended secondary oligonucleotide region of the first probe can include at least 10 copies of the reporter moiety (e.g., at least 50 copies of the reporter moiety, at least 100 copies of the reporter moiety). The reporter moiety can include at least 3 label regions (e.g., at least 4 label regions).

One of the label regions of the reporter moiety can be different from the other label regions of the reporter moiety. Two of the label regions of the reporter moiety can be different from the other label regions of the reporter moiety. The reporter moiety can include at least two different types of label regions, where each different type of label region can include a unique oligonucleotide sequence. The reporter moiety comprises at least three different types of label regions (e.g., at least four different types of label regions). Each of the label regions in the reporter moiety can be a different type of label region. Each label region can include at least 15 nucleotides (e.g., at least 30 nucleotides). Each label region can include a same number of nucleotides.

The methods can include: (a) exposing the sample to a plurality of optical labels, where each of the optical labels includes an oligonucleotide having a sequence, and a species that generates an optical signal; (b) measuring optical signals generated by optical labels of the plurality of optical labels that hybridize to complementary label regions of the reporter moieties; (c) repeating steps (a) and (b) with different pluralities of optical labels; (d) identifying one or more of the reporter moieties in the sample based on the measured optical signals; and (e) determining one or more locations of the RNA in the sample based on the one or more identified reporter moieties. The species that generates the optical signal can be a fluorescent moiety. The species that generates the optical signal can include at least one fluorescent nucleotide.

Measuring optical signals generated by the optical labels can include obtaining at least one image of the optical labels in the sample, and identifying optical signals corresponding to the optical labels in the at least one image. Each plurality of optical labels in step (a) can include a same number of different types of optical labels. Each plurality of optical labels in step (a) can include 3 or more different types of optical labels (e.g., 5 or more different types of optical labels).

The methods can include repeating step (a) until the sample has been exposed to a set of optical labels, where each label region of the reporter moieties has a complementary optical label in the set of optical labels. The methods can include exposing the sample to at least one of the plurality of optical labels more than once. Each time step (a) is performed, each member of the plurality of optical labels in step (a) can include a species that generates a different optical signal. The different optical signals can have different spectral distributions.

Among the plurality of optical labels, at least two of the optical labels can include a common species that generates the optical signal, and the at least two of the optical labels can be exposed to the sample during different repetitions of step (a). Among the plurality of optical labels, first and second optical labels each include a first species that generates the optical signals of the first and second optical labels, third and fourth optical labels each include a second species that generates the optical signals of the third and fourth optical labels, and the first and second species can be different. The optical signals generated by the first and second species can be different.

The methods can include exposing the sample to the first and second optical labels during different repetitions of step (a), and exposing the sample to the third and fourth optical labels during different repetitions of step (a). The methods can include, for at least one of the optical labels, removing the at least one of the optical labels from the sample after measuring an optical signal generated by the at least one of the optical labels. Removing the at least one of the optical labels can include dehybridizing the at least one of the optical labels from one or more label regions of the reporter moieties.

Embodiments of the methods can also include any of the other features described herein, and can include any combination of features that are described in connection with different embodiments, except as expressly stated otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features and advantages will be apparent from the description, drawings, and claims.

### DESCRIPTION OF DRAWINGS

FIG. 1 is a schematic diagram of an example of a probe for RNA in a biological sample.

FIG. 2 is a schematic diagram of an example of a detection moiety.

FIG. 3 is a schematic diagram of an example of a reporter moiety.

FIG. 4 is a schematic diagram of an example of a method for labeling a RNA in a sample.

FIG. 5 is a schematic diagram of another example of a method for labeling a RNA in a sample.

FIG. 6 is a flow chart showing a series of example steps for detecting RNAs in a sample via multiple cycles of hybridization of optical labels.

FIG. 7A is a schematic diagram showing an example set of detection cycles for identifying a RNA in a sample.

FIG. 7B is a schematic diagram showing two different types of RNAs in a sample.

FIG. 7C is a schematic image of the sample of FIG. 7B showing locations of the two different types of RNAs.

FIG. 7D is a schematic diagram showing optical signals measured in each of 6 detection cycles performed on a sample containing both of the different RNAs of FIG. 7B.

FIG. 8 is a schematic diagram of a system for analyzing a biological sample.

FIG. 9 is a schematic diagram of a controller.

Like reference symbols in the various drawings indicate like elements.

### DETAILED DESCRIPTION

### Introduction

Multiplexed fluorescence detection is a widely known technique for detecting proteins, nucleic acids, and other analytes in biological samples. Conventional fluorescence imaging methods involve applying different fluorescent dyes to different analytes in a sample, and then exposing the dyes to incident light to cause each dye to emit fluorescence. By spectrally resolving fluorescence emission signals from the different dyes, analytes in the sample can be identified and quantified. When the fluorescence emission signals are also spatially resolved as in a multispectral fluorescence image, information about the presence and abundance of multiple different analytes at multiple locations in a sample can be obtained. This information is valuable for a wide variety of research and clinical diagnostic purposes, including identifying markers of disease, elucidating intracellular communications pathways, assessing gene expression and cellular regulation activity, and monitoring the effectiveness of pharmaceutical and other therapeutic trials and interventions.

In conventional fluorescence imaging methods, the number of analytes that can be detected can be limited by the number of different measurement signals that can be detected and resolved. Methods exist for separating fluorescence emission signals from dyes that overlap spectrally, even when the overlap is significant. For example, eigenvector decomposition methods such as spectral unmixing can be used to separate such signals, provided that relatively good estimates for the pure spectra of the individual fluorescent dyes (i.e., the eigenvectors) are available or measureable. Even with such techniques, however, the number of different measurement signals that can be resolved—and the corresponding number of different analytes that can be quantified—may be relatively limited due to spectral congestion of the dyes involved.

Limits on the number of different analytes may not represent significant drawbacks for certain types of assays. For example, for certain tumor-specific assays that target a relatively small number of expressed cell markers via antibody-based probes that selectively bind to the markers, the foregoing limits may not represent a significant shortcoming. However, assays that target a more diverse set of cell markers, proteomics assays designed to obtain more general information about expressed proteins in cells, and nucleic acid assays that target cellular RNA and DNA may be designed to quantify tens, hundreds, or thousands of distinct peptides and/or transcripts. For these expansive assays, the above limitations may represent significant roadblocks.

One approach to increasing the number of distinct analytes that can be detected and quantified involves using reporter moieties with spectrally narrow and distinct emission characteristics. For example, quantum dot-based reporter moieties can be synthesized and incorporated into probes that bind specifically to different types of analytes. By controlling the composition and size distribution of the quantum dots, populations of different reporter moieties with (spectrally) closely-spaced but resolvable fluorescence emission signals can be obtained. Incorporated into different types of probes, the resolvable reporter moieties can be used to identify a larger number of different analytes than would typically be possible with conventional fluorescent dye-based reporter moieties.

However, as the number of different analytes increases, the complexity associated with synthesizing populations of distinct reporting moieties increases, as does the time required to synthesize and characterize the reporter moieties prior to use. For an assay involving several hundred different analytes (i.e., peptide or nucleic acid analytes such as RNA transcripts), the synthetic burdens discussed above may be too significant to make such approaches practical and cost-effective.

The methods described herein can be used to detect a wide variety of different RNA species in a sample. Instead of assigning a different “color” to each type of RNA via a distinct reporter moiety that is specifically bound only to RNAs of that type, the methods described herein involve labeling individual RNAs with combinations of different oligonucleotide sequences that are referred to as label regions. Each different type of RNA is labeled with a distinct combination of label regions that form a reporter code. By detecting optical signals generated by optical labels that hybridize to the label regions corresponding to each reporter code, the reporter code of each bound probe in the sample can be identified, and therefore, the RNA to which the probe is bound can be identified.

By labeling RNAs with reporter codes that include combinations of label regions, comparatively fewer individually distinct optical labels are needed to distinguish among a set of different RNAs. As a result, the spectral congestion and difficulties associated with resolving spectrally closely-spaced emission signals can be substantially reduced. As a consequence, a much larger number of different RNAs can be reliably detected and quantified. Further, existing sets of probes that are designed to target particular groups of RNAs can be more readily augmented to include probes for additional RNAs without resorting to complex synthetic methods to produced tightly controlled distributions of spectrally distinct reporter moieties.

### In-Situ Labeling of RNA in Biological Samples

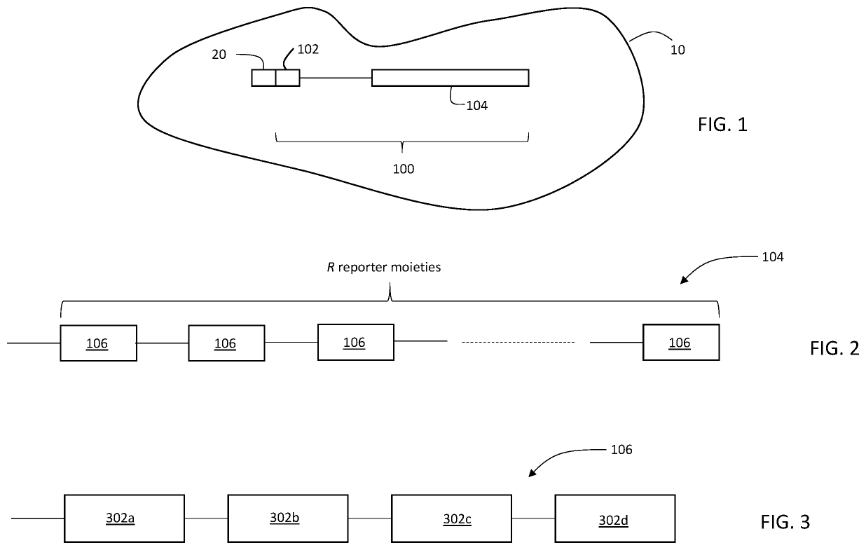

To detect RNA in biological samples, in some embodiments, the sample is exposed to a probe. FIG. 1 is a schematic diagram showing a biological sample 10 that includes an RNA 20. Sample 10 is exposed to a probe 100 that specifically binds to RNA 20. Biological sample 10 can be any one of a variety of different types of samples. Examples of biological sample 10 include, but are not limited to, tissue sections (e.g., fresh sections, fresh-frozen sections, formalin-fixed paraffin embedded sections), tissue biopsies, cells, cell suspensions, cell dispersions, cell cultures, and various bodily fluids such as blood, urine, interstitial fluid, and lymphatic fluid.

In some embodiments, biological sample 10 can be immobilized on a surface. For example, the surface can be a surface of a slide, a plate, a well, a tube, a membrane, or a film. In some embodiments, biological sample 10 can be mounted on a slide. In certain embodiments, biological sample 10 can be fixed using a fixative, such as an aldehyde, an alcohol, an oxidizing agent, a mercurial, a picrate, HOPE fixative, or another fixative. Biological sample 10 may alternatively, or in addition, be fixed using heat fixation. Fixation can also be achieved via immersion or perfusion.

RNA 20 can be any of a variety of different types of RNA. Examples of such RNA types include, but are not limited to, messenger RNA, ribosomal RNA, transfer RNA, transfer-messenger RNA, small nuclear RNA, small nucleolar RNA, signal recognition particle RNA, guide RNA, SmY RNA, Y RNA, antisense RNA, CRISPR RNA, microRNA, small interfering RNA, short hairpin RNA, long noncoding RNA, and enhancer RNA. RNA 20 can contain RNA bases, both DNA and RNA bases, and synthetic bases.

As shown in FIG. 1, probe 100 includes a capture moiety 102 and a detection moiety 104 linked to capture moiety 102. Capture moiety 102 selectively binds to RNA 20 in sample 10 to attach detection moiety 104 to RNA 20. A variety of different reversible and irreversible binding mechanisms can occur between capture moiety 102 and RNA 20. In some embodiments, for example, capture moiety 102 includes an oligonucleotide having a sequence that is at least partially complementary to a sequence of RNA 20. When sample 10 is exposed to probe 100, capture moiety 102 binds to RNA 20 by hybridizing to RNA 20. The binding between capture moiety 102 and RNA 20 can be readily be reversed via dehybridization, e.g., by heating sample 10 and/or introducing one or more chaotropic reagents.

As shown in FIG. 1, capture moiety 102 is linked to detection moiety 104 in probe 100. The linkage between capture moiety 102 and detection moiety 104 can be implemented in various ways. In some embodiments, capture moiety 102 and detection moiety 104 can be linked directly via a covalent or non-covalent bond. That is, capture moiety 102 and detection moiety 104 can be linked directly via a bond, with no intervening moiety or structure between capture moiety 102 and detection moiety 104.

In certain embodiments, capture moiety 102 and detection moiety 104 can be linked via a primary-secondary antibody pair. To label RNA 20 with detection moiety 104, RNA 20 is exposed to a first labeling agent that includes capture moiety 102 conjugated to a primary antibody, which functions as a part of the linkage between capture moiety 102 and detection moiety 104. Once the first labeling agent selectively binds to RNA 20, a second labeling agent is introduced that includes detection moiety 104 conjugated to a secondary antibody that functions as another part of the linkage. The secondary antibody selectively binds to the first antibody, forming a linkage between capture moiety 102 and detection moiety 104 consisting of the associated antibodies, and labeling RNA 20 with detection moiety 104.

In some embodiments, the linkage between capture moiety 102 and detection moiety 104 can be implemented as a double-stranded nucleic acid (e.g., hybridized nucleic acid strands that are at least partially complementary). To label RNA 20 with detection moiety 104, RNA 20 is exposed to a first labeling agent that includes capture moiety 102 linked to a first nucleic acid, which functions as a part of the linkage between capture moiety 102 and detection moiety 104. Once the first labeling agent selectively binds to RNA 20, a second labeling agent is introduced that includes detection moiety 104 linked to a second nucleic acid that functions as another part of the linkage. The second nucleic acid is at least partially complementary to the first nucleic acid, and selectively hybridizes to the first nucleic acid, labeling RNA 20 with detection moiety 104.

In some embodiments, the first nucleic acid can be a nucleic acid sequence that is contiguous with capture moiety 102. In other words, capture moiety 102 and the first nucleic acid can form a continuous nucleic acid sequence in which a portion of the nucleic acid sequence functions as capture moiety 102 (i.e., a capture region), and a portion of the nucleic acid sequence functions as the first nucleic acid (i.e., a linking nucleic acid sequence). The continuous nucleic acid sequence can be single-stranded or double-stranded.

In certain embodiments, the second nucleic acid can be a nucleic acid sequence that is contiguous with detection moiety 104. That is, the second nucleic acid and detection moiety 104 can form a continuous nucleic acid sequence in which a portion of the nucleic acid sequence functions as the second nucleic acid (i.e., a linking nucleic acid region), and a portion of the nucleic acid sequence functions as detection moiety 104 (i.e., a detection region that includes the label regions described herein that form detection moiety 104). The continuous nucleic acid sequence can be single-stranded or double-stranded.

In some embodiments, capture moiety 102 can be linked to the first nucleic acid through conjugation, e.g., capture moiety 102 can be covalently bonded to the first nucleic acid. Any of a wide variety of different linkages can be used to covalently bond capture moiety 102 and the first nucleic acid, as discussed below. In certain embodiments, detection moiety 104 can be linked to the second nucleic acid through covalent bonding, using any of the different linkages described herein.

In some embodiments, the first and second nucleic acids that function as portions of the linkage between capture moiety 102 and detection moiety 104 do not directly hybridize. Instead, the first and second nucleic acids each hybridize to a portion of a bridging oligonucleotide that includes nucleic acid sequences that are at least partially complementary to each of the first and second nucleic acids. Bridging oligonucleotides can be single-stranded, such that a portion of the bridging oligonucleotide hybridizes to the first nucleic acid and a portion of the bridging oligonucleotide hybridizes to the second nucleic acid. Bridging oligonucleotides can be partially double-stranded, with overhangs on one or both strands that hybridize to the first and second nucleic acids to form the linkage between capture moiety 102 and detection moiety 104.

Bridging oligonucleotides can be linear such that a single capture moiety is linked to a single reporter moiety. Alternatively, bridging oligonucleotides can be branched, and can include a single nucleic acid sequence that hybridizes to capture moiety 102, and multiple nucleic acid sequences that hybridize to detection moieties 104. As a result, a single capture moiety 102 can be linked to multiple detection moieties 104, allowing for amplification of optical signals that correspond to the RNA to which capture moiety 102 selectively binds.

In certain embodiments, the linkage between capture moiety 102 and detection moiety 104 can be implemented as any of a variety of aliphatic and/or aromatic linking species. Further, as discussed above, in some embodiments, capture moiety 102 can be covalently bonded to the first nucleic acid, either via a direct covalent bond, or via any of a variety of aliphatic and/or aromatic linking species. Further still, as discussed above, in certain embodiments, detection moiety 104 can be covalently bonded to the second nucleic acid, either via a direct covalent bond, or via any of a variety of aliphatic and/or aromatic linking species.

Examples of linking species that can be used to directly link capture moiety 102 and detection moiety 104, to link capture moiety 102 and the first nucleic acid, and/or to link detection moiety 104 and the second nucleic acid, include—but are not limited to— C₁₋₂₀ cyclic and non-cyclic alkyl species, C₂₋₂₀ cyclic and non-cyclic alkene species, C₂₋₂₀ cyclic and non-cyclic alkyne species, and C₃₋₂₄ aromatic species. Any of the foregoing species can include heteroatoms such as, but not limited to, O, S, N, and P. Any of the foregoing species can also include one or more substituents selected from the group consisting of: halide groups; nitro groups; azide groups; hydroxyl groups; primary, secondary, and tertiary amine groups; aldehyde groups; ketone groups; amide groups; ether groups; ester groups; thiocyanate groups; and isothiocyanate groups.

In general, each detection moiety 104 includes one or more reporter moieties 106 that include oligonucleotide sequences that selectively hybridize to different optical labels which generate optical signals that can be used to identify the reporter moieties. FIG. 2 is a schematic diagram showing an example of a detection moiety 104 that includes R reporter moieties 106. In general, as will be described in greater detail below, each of the reporter moieties contains the same unique combination of label regions that is uniquely associated with the reporter moiety, and with the RNA 20 that selectively binds to capture moiety 102 linked to detection moiety 104. In some embodiments, each of the R reporter moieties 106 are the same. In certain embodiments, some of the reporter moieties differ from one another, but in general, each reporter moiety still includes the same unique combination of label regions that is associated with the RNA 20 that binds to capture moiety 102.

Detection moiety 104 can generally include any number R of reporter moieties 106. In some embodiments, for example, R is 1 or more (e.g., 2 or more, 3 or more, 5 or more, 7 or more, 10 or more, 15 or more, 20 or more, 25 or more, 30 or more, 35 or more, 40 or more, 50 or more, 60 or more, 70 or more, 100 or more, 120 or more, 150 or more, 200 or more, 300 or more, 400 or more, 500 or more, 700 or more, 1000 or more, 1200 or more, 1400 or more, 1600 or more, 1800 or more, 2000 or more, 2500 or more, 3000 or more, 4000 or more, 5000 or more, 7000 or more, 10000 or more, or even more). Each of the reporter moieties 106 in detection moiety 104 can generally and independently have any of the properties and features described herein in connection with reporter moieties.

When detection moiety 104 contains multiple reporter moieties 106, the optical signal that is generated when optical labels hybridize to the multiple reporter moieties is generally more intense than optical signals that arise from single optical labels. The higher intensity optical signals allow low concentration RNAs to be detected, and even single RNA molecules can be detected in this manner.

As discussed above, each detection moiety 104 includes one or more reporter moieties 106. FIG. 3 is a schematic diagram showing an example of a reporter moiety 106 of detection moiety 104. Reporter moiety 106 includes multiple label regions. In general, each label region is an oligonucleotide sequence consisting of multiple nucleotide bases. The oligonucleotide sequence of a label region is at least partially complementary to an oligonucleotide sequence of an optical label. Hybridization of the optical label to the label region localizes the optical label in the sample at the location of the label region, as will be described in greater detail below.

In the example of FIG. 3, reporter moiety 106 includes four label regions 302a-302d. More generally, however, reporter moiety 106 includes two or more (e.g., three or more, four or more, five or more, six or more, seven or more, eight or more, 10 or more, 15 or more, 20 or more, or even more) label regions.

In some embodiments, each of the label regions in reporter moiety 106 is different (i.e., has a different nucleic acid sequence). Alternatively, in certain embodiments, one or more of the label regions in reporter moiety 106 has a sequence that is common to another one of the label regions in reporter moiety 106. The number of label regions that are the same in a reporter moiety can be 2 or more (e.g., 3 or more, 4 or more, 5 or more, 6 or more, 7 or more, 8 or more). Label regions that are the same in a reporter moiety 106 can be used for signal amplification and/or to compensate for mis-hybridization of optical labels and/or the absence of hybridization of optical labels.

In some embodiments, each label region includes multiple nucleotide bases forming a nucleic acid sequence. The label regions of a reporter moiety 106 can each have the same number of nucleotides, or one or more of the label regions can have a different number of nucleotides. In certain embodiments, each of the label regions has the same number of nucleotides. In some embodiments, each of the label regions has a different number of nucleotides.

Each label region of reporter moiety 106 includes a nucleic acid sequence. Each label region can independently include DNA bases (e.g., A, C, G, T), RNA bases (e.g., A, C, G, U), and any combination of DNA and/or RNA bases. Each label region can also include non-natural (e.g., synthetic) nucleotides, including DNA analogues and/or RNA analogues. Examples of such synthetic analogues include, but are not limited to, peptide nucleic acids (PNAs), morpholino and locked nucleic acids (LNAs), glycol nucleic acids, and threose nucleic acids.

The length of a label region (e.g., the number of nucleotides in a label region) can generally be selected as desired to ensure efficient and selective hybridization with an optical label. In some embodiments, each label region can include at least 5 (e.g., at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100) nucleotides.

In certain embodiments, each label region can have between 5-30, between 5-25, between 5-20, between 10-20, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides.

In some embodiments, each label region can have no more than 5 (e.g., no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100) nucleotides.

In some embodiments, each label region can be fully single stranded. Alternatively, in certain embodiments, one or more label regions can be at least partially double stranded. A partially double stranded portion of a label region can be at the 3′ end of the label region, at the 5′ end of the label region, or between the 5′ end and 3′ ends of the label region.

In some embodiments, the reporter moieties 106 of the detection moiety 104 of each probe 100 bound to an RNA in sample 10 include the same number of label regions. In certain embodiments, however, the reporter moieties 106 of the detection moiety 104 of at least one (e.g., two or more, three or more, five or more, 10 or more, 20 or more, 30 or more, 50 or more, 100 or more) probes 100 bound to an RNA in sample 10 include a different number of label regions from the reporter moieties 106 of the detection moieties 104 of the other probes 100 bound to RNAs in the sample. In certain embodiments, sample 10 can include 2 or more (e.g., 3 or more, 4 or more, 5 or more, 6 or more, or even more) groups of probes, where the reporter moieties of the detection moieties of the probes within each group have a different number of label regions from the reporter moieties of the detection moieties of the probes in the other groups.

In some embodiments, sample 10 can be directly exposed to probe 100 to label RNA 20 with a detection moiety 104 that includes one or reporter moieties 106, each of which includes one or more label regions, as discussed above. Capture moiety 102 selectively binds to RNA 20 (e.g., via hybridization), labeling RNA 20 with probe 100. Probe 100, and specifically detection moiety 104 and reporter moieties 106, can have any of the properties discussed above. This type of direct labeling of RNA 20 with probe 100 represents one type of workflow for labeling RNA 20 prior to detection.

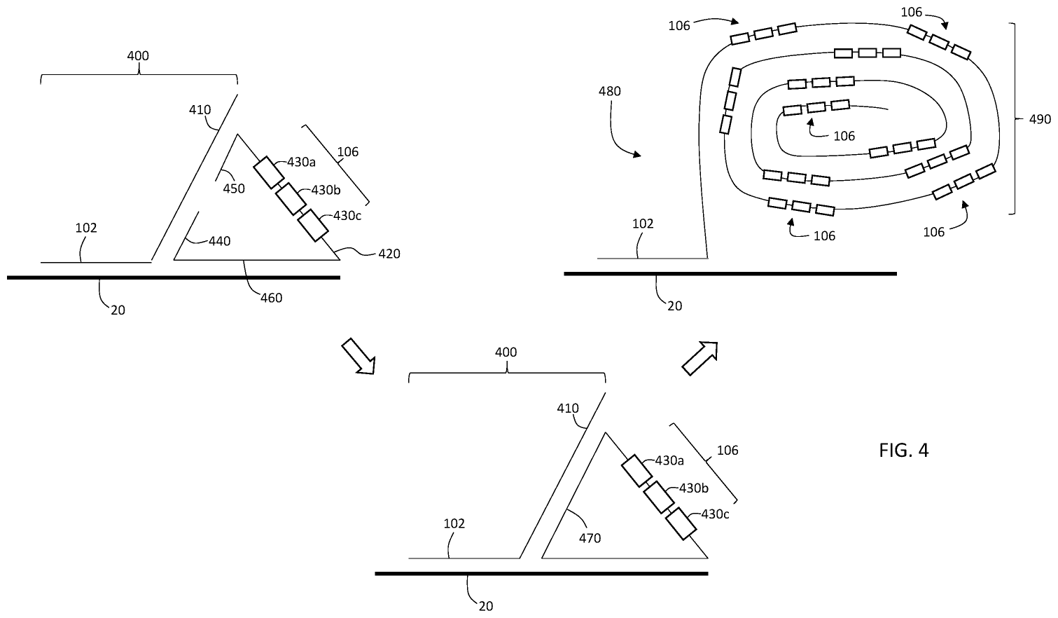

Other methods for labeling RNA 20 with probe 100 can also be used. In some embodiments, for example, detection moiety 104 can be formed (e.g., by nucleic acid synthesis, by extension of one or more nucleic acids, and/or by altering one or more nucleic acids) in situ following hybridization of a first probe to RNA 20 in sample 10. FIG. 4 is a schematic diagram showing an example of such a workflow for labeling RNA 20 with a probe.

In a first step of the workflow shown in FIG. 4, RNA 20 in a sample is contacted with a first probe 400. First probe 400 includes a capture moiety 102 and a secondary oligonucleotide region 410. As shown in FIG. 4 and discussed above, capture moiety 102 has an oligonucleotide sequence that is at least partially complementary to RNA 20, such that capture moiety 102 hybridizes to RNA 20. In general, secondary oligonucleotide region 410 is not complementary to RNA 20. As a result, secondary oligonucleotide region 410 does not hybridize to RNA 20, and instead extends freely away from RNA 20.

Secondary oligonucleotide region 410 can generally include any number of nucleotides. In some embodiments, for example, secondary oligonucleotide region 410 includes 10 or more (e.g., 20 or more, 30 or more, 40 or more, 50 or more, 60 or more, 70 or more, 80 or more, 100 or more, 120 or more, 140 or more, 160 or more, 180 or more, 200 or more, 250 or more, 300 or more, 400 or more, 500 or more, or even more) nucleotides.

In a second step of the workflow shown in FIG. 4, the sample is contacted with a second probe 420. Second probe 420 includes a reporter moiety 106 with a plurality of label regions. In the example of FIG. 4, second probe 420 includes a reporter moiety 106 that includes three label regions 430a-430c. More generally, however, the reporter moiety 106 of second probe 420 can include two or more (e.g., three or more, four or more, five or more, six or more, seven or more, eight or more, 10 or more, 12 or more, 15 or more, 20 or more, 25 or more, or even more) label regions. Reporter moiety 106 of second probe 420 can have any of the properties of the reporter moieties 106 described herein.

Second probe 420 also includes first probe binding region 440 and second probe binding region 450. As shown in FIG. 4, first probe binding region 440 has an oligonucleotide sequence that is at least partially complementary to a portion of secondary oligonucleotide region 410. Further, second probe binding region 450 has an oligonucleotide sequence that is at least partially complementary to another portion of secondary oligonucleotide region 410. As a result, the first and second probe binding regions each hybridize to different portions of secondary oligonucleotide region 410, in relatively close proximity to one another.

In some embodiments, the hybridization region between first probe binding region 440 and a portion of secondary oligonucleotide region 410 is 10 nucleotides or more (e.g., 15 nucleotides or more, 20 nucleotides or more, 25 nucleotides or more, 30 nucleotides or more, 40 nucleotides or more, 50 nucleotides or more, 60 nucleotides or more, 70 nucleotides or more, 80 nucleotides or more, 90 nucleotides or more, 100 nucleotides or more, or even more). In general, the hybridization region between second probe binding region 450 and a portion of secondary oligonucleotide region 410 can be any of the lengths discussed above in connection with the hybridization region between first probe binding region 440 and a portion of secondary oligonucleotide region 410. The two hybridization regions can be the same length or different lengths.

In certain embodiments, the portion of secondary oligonucleotide region 410 between the two hybridization regions can be one or more nucleotides (e.g., two or more nucleotides, three or more nucleotides, four or more nucleotides, five or more nucleotides, six or more nucleotides, eight or more nucleotides, 10 or more nucleotides, 12 or more nucleotides, 14 or more nucleotides, 16 or more nucleotides, 18 or more nucleotides, 20 or more nucleotides, or even more nucleotides.

In some embodiments, second probe 420 hybridizes only to secondary oligonucleotide region 410, e.g., via first probe binding region 440 and second probe binding region 450. In certain embodiments, however, second probe 420 also includes an optional target binding region 460. As shown in FIG. 4, target binding region 460 consists of an oligonucleotide sequence that is at least partially complementary to a portion of RNA 20, so that target binding region 460 hybridizes to a portion of RNA 20 in relative proximity to the portion of RNA 20 to which first probe 410 hybridizes.

In general, the hybridization region between target binding region 460 and a portion of RNA 20 is 1 nucleotide or more (e.g., 2 nucleotides or more, 3 nucleotides or more, 4 nucleotides or more, 5 nucleotides or more, 6 nucleotides or more, 8 nucleotides or more, 10 nucleotides or more, 15 nucleotides or more, 20 nucleotides or more, 25 nucleotides or more, 30 nucleotides or more, 40 nucleotides or more, 50 nucleotides or more, 60 nucleotides or more, 70 nucleotides or more, 80 nucleotides or more, 90 nucleotides or more, 100 nucleotides or more, or even more) in length.

Along the length of RNA 20, two hybridization regions are present: between capture moiety 102 and a first portion of RNA 20, and between target binding region 460 and a second portion of RNA 20. In some embodiments, the number of nucleotides in the sequence of RNA 20 between the two hybridization regions can be zero or more nucleotides (e.g., one or more nucleotides, two or more nucleotides, three or more nucleotides, four or more nucleotides, five or more nucleotides, six or more nucleotides, eight or more nucleotides, 10 or more nucleotides, 12 or more nucleotides, 14 or more nucleotides, 16 or more nucleotides, 18 or more nucleotides, 20 or more nucleotides, or even more nucleotides.

Referring again to FIG. 4, the next step in the workflow is to ligate the ends of the first and second probe binding regions 440 and 450 to form a closed nucleic acid construct 470 that remains hybridized to first probe 400. Thereafter, first probe 400 is extended from the tail of secondary oligonucleotide region 410 by performing rolling circle amplification (RCA), with closed nucleic acid construct 470 functioning as the template for extension of secondary oligonucleotide region 410.

To perform ligation, a ligase (e.g., an enzyme that joins the ends of a single oligonucleotide, or joins the ends of two different oligonucleotides) is introduced into the sample. The enzyme joins the 5′ end of an oligonucleotide (which is typically phosphorylated) to the 3′ end of the oligonucleotide (or to the 3′ end of another oligonucleotide). In FIG. 4, for example, the 5′ end of second probe 420 can be phosphorylated, and joined by the ligase to the 3′ end of second probe 420. A variety of different ligases can be used for this purpose including, but not limited to, AT-dependent double-strand polynucleotide ligases, NAD+-dependent double-strand DNA and RNA ligases, and single-strand polynucleotide ligases. Examples of such ligases include bacterial ligases (e.g., E. coli DNA ligase, Tag DNA ligase, Ampligase® DNA ligase, T3 DNA ligase, T4 DNA ligase, and T7 ligase.

To perform RCA, a polynucleotide polymerase is introduced, along with dNTP precursors and cofactors. Secondary oligonucleotide region 410 is then extended, with construct 470 functioning as the template for the extension reaction. A wide variety of different polymerases can be used. One such polymerase is 129 DNA polymerase. Methods and reagents for performing RCA are described, for example, in the following references, the entire contents of each of which are incorporated by reference herein: Lizardi et al., Nat. Genetics 19: 225-232 (1998); Ali et al., Chem. Soc. Rev. 43: 3324 (2014); Baner et al., Nucleic Acids Res. 26: 5073-5078 (1998); Dean et al., Genome Res. 11: 1095-1099 (2001); Schweitzer et al., Nat. Biotech. 20: 359-365 (2002); U.S. Pat. Nos. 6,054,274; 6,291,187; 6,323,009; 6,344,329; and 6,368,801.

In general, any number of RCA cycles can be performed to extend secondary oligonucleotide region 410. For example, in some embodiments, the number of RCA cycles is 2 or more (e.g., 3 or more, 4 or more, 5 or more, 6 or more, 7 or more, 8 or more, 10 or more, 12 or more, 14 or more, 16 or more, 18 or more, 20 or more, 25 or more, 30 or more, 40 or more, 50 or more, or even more).

Because the label regions are present in reporter moiety 106 of nucleic acid construct 470 which functions as the template for RCA, each cycle of amplification introduces another copy of the reporter code into the extended secondary oligonucleotide region 410. As a result, after n cycles, the number of copies of the reporter code in the extended secondary oligonucleotide region is n. In certain embodiments, n can be 2 or more (e.g., 3 or more, 4 or more, 5 or more, 6 or more, 7 or more, 8 or more, 10 or more, 12 or more, 14 or more, 16 or more, 18 or more, 20 or more, 25 or more, 30 or more, 40 or more, 50 or more, 60 or more, 70 or more, 80 or more, 100 or more, 200 or more, 300 or more, 400 or more, 500 or more, 600 or more, 800 or more, 1000 or more, 1500 or more, 2000 or more, 3000 or more, 4000 or more, 5000 or more, 6000 or more, 8000 or more, 10,000 or more, 15,000 or more, 20,000 or more, 25,000 or more, or even more).

After RCA is terminated, a modified first probe 400 remains hybridized to RNA 20. As shown in FIG. 4, the modified first probe includes an extended secondary oligonucleotide region 410 that includes multiple copies of the reporter moiety 106 in nucleic acid construct 470, and therefore, multiple copies of the set of label regions of reporter moiety 106.

Following extension of secondary oligonucleotide region 410 as shown in FIG. 4, RNA 20 is bound to a probe 480 that includes capture moiety 102 and a detection moiety 490. The detection moiety 490 includes the extended secondary oligonucleotide region 410 with multiple reporter moieties 106, each of which includes the set of label regions that were introduced as part of second probe 420.

As shown in FIG. 4, in some embodiments, probe 480 is attached to RNA 20 through capture moiety 102, which binds RNA 20 (i.e., via hybridization to all or a portion of RNA 20). In certain embodiments, probe 480 can also be bound to the sample (i.e., to RNA 20, or to another portion of the sample) in another manner.

In some embodiments, probe 480 can be bound to the sample via one or more covalent bonds. In certain embodiments, the covalent bonds can be formed by cross-linking one or more portions of probe 480 to the sample. A variety of different methods can be used to cross-link probe 480 to the sample.

For example, in some embodiments, during extension of secondary oligonucleotide region 410 to form detection moiety 490, the extension reaction can be performed by incorporating 5-(3-aminoallyl)-dUTP into the extended secondary oligonucleotide region 410. The extended secondary oligonucleotide region 410 can then be cross-linked to the sample by introducing a bifunctional NHS ester (such as, for example, PEGylated bis(sulfosuccinimydl)suberate (BS(PEG)9)). Cross-linking can be performed during extension of the secondary oligonucleotide region 410, or after the extension reaction is complete. Additional aspects of these methods and other cross-linking methods are described, for example, in U.S. Patent Application Publication No. US 2018/0051322, the entire contents of which are incorporated herein by reference.

A variety of other methods can also be used to cross-link or otherwise fix probe 480 in position relative to the sample. Such methods include, but are not limited to, linking through cell surface functional groups and DNA-binding proteins. Features and aspects of such methods are described, for example, in U.S. Pat. Nos. 9,327,036 and 10,253,333, the entire contents of each of which are incorporated by reference herein.

Additional features and aspects of the workflow shown in FIG. 4 are described, for example, in U.S. Patent Application Publication No. US 2019/0055594, the entire contents of which are incorporated herein by reference.

In the foregoing description, second probe 420 is a linear oligonucleotide with first and second probe binding regions 440 and 450 that hybridize to different oligonucleotide sequences of secondary oligonucleotide region 410 in end-to-end fashion, spaced closely enough such that the first and second probe binding regions can be ligated together to form a circularized nucleic acid construct 470. In some embodiments, circularized nucleic acid construct 470 is introduced directly into the sample. The circularized nucleic acid construct 470 includes a probe binding region that hybridizes to a portion of secondary oligonucleotide region 410, and a reporter moiety 106 as described above. One advantage of introducing circularized nucleic acid construct 470 is that the ligation step can be eliminated, and potential cross-hybridization involving probe binding regions 440 and/or 450 with nucleotides other than secondary oligonucleotide region 410 can be reduced or eliminated.

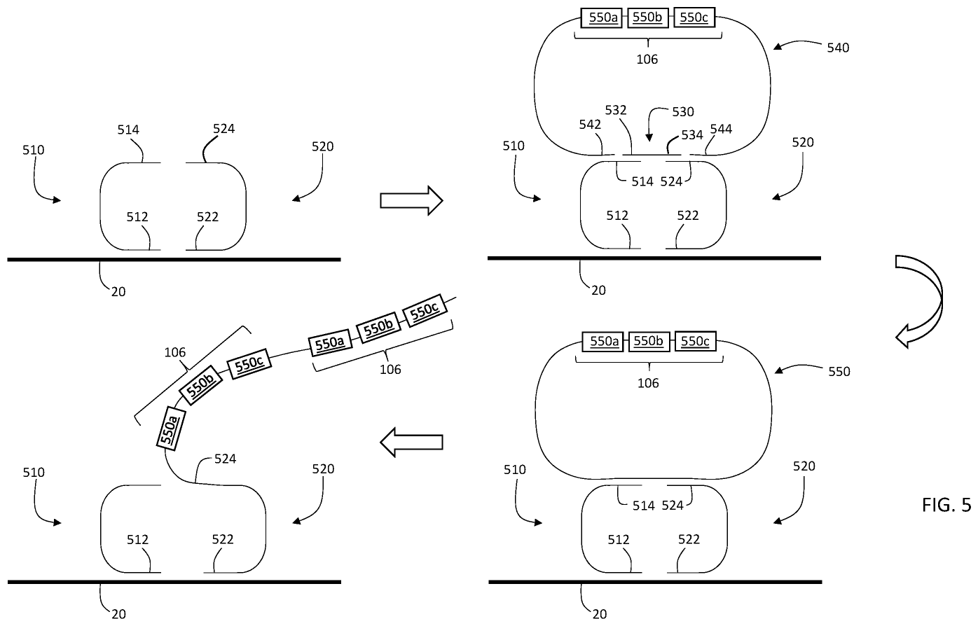

In some embodiments, RCA can be used with two probes that selectively bind to different regions of RNA 20 to generate a probe in situ that includes a detection moiety 104 as described herein. FIG. 5 is a schematic diagram showing an example of a workflow that implements such a strategy. In FIG. 5, sample 10 containing RNA 20 is first exposed to two probes 510 and 520. Probes 510 and 520 each include target binding regions 512 and 522, respectively, that are complementary to different portions of RNA 20, and therefore selectively hybridize to those respective portions of RNA 20. Probes 510 and 520 each also include secondary regions 514 and 524, respectively, that are not complementary to RNA 20, and therefore extend freely away from RNA 20 as shown in FIG. 5.

In general, each of the target binding regions 512 and 522 function as capture moieties, and can have any of the properties discussed herein in connection with capture moiety 102. Further, secondary regions 514 and 524 function in a manner similar to secondary oligonucleotide region 410 in FIG. 4, and can have any of the properties discussed herein in connection with that region.

Probes 510 and 520 can have the same number of nucleotides, or a different number of nucleotides. In general, each probe can include 10 or more (e.g., 15 or more, 20 or more, 25 or more, 30 or more, 35 or more, 40 or more, 45 or more, 50 or more, 60 or more, 70 or more, 80 or more, 100 or more, 120 or more, 150 or more, 200 or more, 300 or more, 400 or more, 500 or more, 600 or more, 800 or more, 1000 or more, or even more) nucleotides.

The oligonucleotide sequences of target binding regions 512 and 522 are generally selected such that probes 510 and 520, when hybridized to RNA 20, are located in relatively close proximity. In some embodiments, for example, the number of nucleotides of RNA 20 between the region of RNA 20 that binds to target binding region 512 and the region of RNA 20 that binds to target binding region 522 is 1 or more (e.g., 2 or more, 3 or more, 4 or more, 5 or more, 6 or more, 8 or more, 10 or more, 12 or more, 14 or more, 16 or more, 18 or more, 20 or more, or even more). The number of nucleotides of RNA 20 between the region of RNA 20 that binds to target binding region 512 and the region of RNA 20 that binds to target binding region 522 can be 30 or less (e.g., 28 or less, 26 or less, 24 or less, 22 or less, 20 or less, 18 or less, 16 or less, 14 or less, 12 or less, 10 or less, 9 or less, 8 or less, 7 or less, 6 or less, 5 or less, 4 or less, 3 or less, 2 or less, 1, or even 0).

Following binding of probes 510 and 520, in the next step of the workflow shown in FIG. 5, third probe 530 and fourth probe 540 are introduced. Third probe 530 functions as a splint probe. A first portion 532 of third probe 530 is an oligonucleotide having a sequence that is complementary to a portion of secondary region 514 of first probe 510. As a result, the first portion 532 selectively hybridizes to secondary region 514. A second portion 534 of third probe 530 is an oligonucleotide having a sequence that is complementary to a portion of secondary region 524 of second probe 520. The second portion 534 selectively hybridizes to secondary region 524, so that third probe 530 is hybridized to both probes 510 and 520, bridging the gap between the two probes as shown in FIG. 5.

Fourth probe 540 contains a first portion 542 that is an oligonucleotide having a sequence that is complementary to a portion of secondary region 514 of first probe 510 that is near to the portion of secondary region 514 that is complementary to first portion 532 of third probe 530. As a result, first portion 542 hybridizes to secondary region 514 of first probe 510 at a location near to first portion 532 of third probe 530. Similarly, fourth probe 540 contains a second portion 544 that is an oligonucleotide having a sequence that is complementary to a portion of secondary region 524 of second probe 520 that is near to the portion of secondary region 524 that is complementary to second portion 534 of third probe 530. As a result, second portion 544 hybridizes to secondary region 524 of second probe 520 at a location near to second portion 534 of third probe 530.

In some embodiments, the number of nucleotides of secondary region 514 between the regions that bind to regions 532 and 542 of third and fourth probes, respectively, can be 1 or more (e.g., 2 or more, 3 or more, 4 or more, 5 or more, 6 or more, 7 or more, 8 or more, 9 or more, 10 or more, 12 or more, 14 or more, 16 or more, 18 or more, 20 or more, or even more). The number of nucleotides of secondary region 514 between the regions that bind to regions 532 and 542 of third and fourth probes, respectively, can be 30 or less (e.g., 28 or less, 26 or less, 24 or less, 22 or less, 20 or less, 18 or more, 16 or less, 14 or less, 12 or less, 10 or less, 9 or less, 8 or less, 7 or less, 6 or less, 5 or less, 4 or less, 3 or less, 2 or less, 1, or even 0).

In certain embodiments, the number of nucleotides of secondary region 524 between the regions that bind to regions 534 and 544 of third and fourth probes, respectively, can be 1 or more (e.g., 2 or more, 3 or more, 4 or more, 5 or more, 6 or more, 7 or more, 8 or more, 9 or more, 10 or more, 12 or more, 14 or more, 16 or more, 18 or more, 20 or more, or even more). The number of nucleotides of secondary region 524 between the regions that bind to regions 534 and 544 of third and fourth probes, respectively, can be 30 or less (e.g., 28 or less, 26 or less, 24 or less, 22 or less, 20 or less, 18 or more, 16 or less, 14 or less, 12 or less, 10 or less, 9 or less, 8 or less, 7 or less, 6 or less, 5 or less, 4 or less, 3 or less, 2 or less, 1, or even 0).

In some embodiments, the length of third probe 530 can be 4 or more nucleotides (e.g., 6 or more, 8 or more, 10 or more, 12 or more, 14 or more, 16 or more, 18 or more, 20 or more, 25 or more, 30 or more, 40 or more, 50 or more, 60 or more, 80 or more, 100 or more, or even more) nucleotides.

In certain embodiments, the length of fourth probe 540 can be 10 or more nucleotides (e.g., 15 or more, 20 or more, 25 or more, 30 or more, 35 or more, 40 or more, 45 or more, 50 or more, 55 or more, 60 or more, 65 or more, 70 or more, 75 or more, 80 or more, 90 or more, 100 or more, 120 or more, 140 or more, 160 or more, 180 or more, 200 or more, 300 or more, 400 or more, 500 or more, 600 or more, 800 or more, 1000 or more, or even more) nucleotides.

Fourth probe 540 generally functions in a manner similar to second probe 420 in FIG. 4. In addition to regions 542 and 544, fourth probe 540 includes a reporter region 106 that includes label regions 550a-550c. Reporter region 106 can generally have any of the properties of the reporter regions discussed herein. Although reporter region 106 is shown by way of example in FIG. 5 as having three label regions 550a-550c, more generally reporter region 106 can include any number of label regions as discussed herein. Each of the label regions can have any of the properties of label regions discussed herein.

Following hybridization of the third and fourth probes in FIG. 5, the third and fourth probes are ligated to form a circularized nucleic acid construct 550. Ligation can generally be performed as described above in connection with FIG. 4. Nucleic acid construct 550 includes reporter moiety 106 from fourth probe 540, and remains hybridized to first and second probes 510 and 520.

Then, RCA is performed to extend one of first and second probes 510 and 520, using nucleic acid construct 550. RCA proceeds as described above in connection with FIG. 4, and yields an extended probe with multiple copies of reporter 106 in the extended nucleic acid. In FIG. 5, secondary region 524 has been extended using nucleic acid construct 550 as a template. As a result, secondary region 524 includes multiple reporter moieties 106, each of which is the same as reporter moiety 106 of construct 550 (and fourth probe 540).

In effect, target binding region 522 of second probe 520 functions as a capture moiety (i.e., capture moiety 102), while the extended secondary region 524 of second probe 520 functions as a detection moiety (i.e., detection moiety 104). Second probe 520 remains hybridized to RNA 20 in the sample, which allows for detection of RNA 20 as will be discussed further. Although in FIG. 5 second probe 520 has been extended via RCA, in some embodiments, first probe 510 can instead be extended via RCA, as the first and second probes are, in effect, interchangeable with respect to one another.

In the foregoing description, fourth probe 540 is a linear oligonucleotide and third probe 530 functions as a bridging oligonucleotide that is ligated to the ends of fourth probe 540 to form a circularized nucleic acid construct. In some embodiments, instead of introducing third and fourth probes 530 and 540 into the sample and ligating the probes, circularized nucleic acid construct 550 can be introduced directly into the sample. The circularized nucleic acid construct 550 includes a probe binding region that hybridizes to a portion of secondary region 524 of second probe 520 (and/or to a portion of secondary region 514 of first probe 510), and then RCA is performed as described above to generate an extended first or second probe. As discussed previously, one advantage of introducing circularized nucleic acid construct 550 directly is that the ligation step can be eliminated, and potential cross-hybridization among probes can be reduced or eliminated.

Further aspects of the foregoing methods are described, for example, in U.S. Patent Application Publication No. US 2016/0108458, the entire contents of which are incorporated by reference herein.

It should be noted that while the foregoing discussions focus on the labeling of a single RNA 20 with a single probe 100, more generally the methods described herein can be used to label multiple different RNAs in sample 10 concurrently. Furthermore, the methods described herein can be used to label multiple portions of the same RNA sequentially or concurrently.

To detect multiple different RNAs, and/or to label multiple portions of a RNA, sample 10 is exposed to multiple pluralities of probes, where each plurality of probes includes a different capture moiety 102 that selectively binds to a different RNA or to a different portion of the same RNA in sample 10. Each plurality of probes represents a different type of probe that selectively binds to a different type of RNA or to a different portion of the same RNA in sample 10.

For a workflow in which probes 100 have been synthesized prior to exposure of the sample to the probes, a composition is prepared that contains populations of different types of probes. For each probe type, the members of the corresponding population of probes each have a common capture moiety 102 that selectively binds to a corresponding type of RNA 20 and/or to a corresponding portion of a type of RNA 20 (i.e., a RNA with a specific oligonucleotide sequence), and a detection moiety 104 that includes reporter moieties that each have a common set of label regions. In some embodiments, the set of label regions is the same among all reporter moieties of the probe type. In certain embodiments, all reporter moieties of the probe type contain the same set of label regions, and certain reporter moieties can also include additional label regions and/or other structural features.

In some embodiments, the composition includes more than one probe type that binds to the same type of RNA. For example, the composition can include a first probe type that binds to a first portion of a type of RNA (i.e., a RNA transcript having a particular oligonucleotide sequence), and a second probe type that binds to a second portion of the same type of RNA. Detection moieties of the first and second probe types can be the same or different.

More generally, for a particular type of RNA, the composition can include one or more (e.g., two or more, three or more, four or more, five or more, six or more, seven or more, eight or more, nine or more, ten or more, or even more) different types of probes, each of which binds to a different portion (e.g., a different oligonucleotide sequence) of the type of RNA. Each of the different types of probes that binds to the same type of RNA can have a different type of capture moiety 102 (i.e., a capture moiety 102 that binds to a different portion of the oligonucleotide sequence of RNA 20). Among the different types of the capture probes, the capture moieties can have the same or different numbers of nucleotides, and the capture moieties can individually have any of the properties discussed herein in connection with capture moieties.

In some embodiments, among the different types of probes that bind different portions of the same type of RNA, the detection moieties 104 are the same. That is, the composition includes two or more different types of probes, each of which binds to a different portion of the same type of RNA 20, and each of which includes the same detection moieties (i.e., the same number and type of reporter moieties 106 in the detection moieties 104). Alternatively, the composition can include two or more different types of probes, each of which binds to a different portion of the same type of RNA 20, and each of which includes the detection moieties with the same type of reporter moieties 106, but the number of reporter moieties 106 in some or all of the different types of detection moieties 104 can be different.

In certain embodiments, one or more of the detection moieties among the different types of probes are different from the other detection moieties of the different probes. For example, in some embodiments, detection moieties differ according to the number of reporter moieties 106 in the detection moieties. In certain embodiments, detection moieties differ according to the type of reporter moieties 106 present in the detection moieties. In some embodiments, detection moieties differ according to both the number and type of reporter moieties 106 present in the detection moieties. In general, the reporter moieties 106 in the detection moieties can individually have any of the properties of reporter moieties discussed herein.

For the workflow shown in FIG. 4, the sample can be exposed to a first composition that contains multiple pluralities of first probes 400. Each plurality of first probes 400 is a different type of probe that selectively hybridizes to a different type of RNA 20 (i.e., a RNA with a different oligonucleotide sequence) or to a different portion of the same type of RNA 20 to which another probe of first probes 400 binds (i.e., a portion of RNA 20 with a different oligonucleotide sequence from the portion of RNA 20 to which another probe binds).

Then, the sample is exposed to a second composition that includes multiple pluralities of second probes 420. In some embodiments, each plurality of second probes 420 is a different type of second probe 420, and selectively hybridizes to a different type of first probe 400. Alternatively, in certain embodiments, where the first plurality of probes includes multiple types of probes 400 that bind to different portions of the same type of RNA 20, one type of second probe 420 can hybridize to each of the multiple types of probes 400 that bind to different portions of the same type of RNA 20.

In some embodiments, where the first plurality of probes includes multiple groups of different first probe types such that each group of different first probe types includes multiple types of first probes 400 that bind to different portions of the same type of RNA 20 (i.e., a different type of RNA 20 for each group of first probe types), one type of second probe 420 can hybridize to each of the different first probe types 400 in a first group of different first probe types, a second type of second probe 420 can hybridize to each of the different first probe types 400 in a second group of different first probe types, a third type of second probe 420 can hybridize to each of the different first probe types 400 in a third group of different first probe types, and so on.

According to the foregoing workflow, in some embodiments, each different type of reporter moiety 106 that is introduced via the second probes 420 can be selectively associated with only one type of RNA 20, either by selective hybridization of the corresponding second probe 420 to only one type of first probe 400, or by hybridization of the corresponding second probe 420 to multiple types of first probes 400, each of which binds to a different portion of the same type of RNA 20.

For the workflow shown in FIG. 5, the sample can be exposed to a first composition that contains multiple pluralities of first probes 510 and second probes 520. For a particular plurality of first probes 510, the composition can include a counterpart plurality of second probes 520, such that the first probes and second probes selectively hybridize to a particular RNA species in the sample. As such, each plurality of first probes 510 is a different type of first probe that selectively hybridizes to a different type of RNA 20, and each counterpart plurality of second probes 520 is a different type of second probe that selectively hybridizes to the type of RNA 20 that hybridizes to the type of first probe. The sample can then be exposed to pluralities of third probes 530 and fourth probes 540.

In some embodiments, following exposure of the sample to the first composition, the sample is exposed to a second composition that includes only third probes 530. In certain embodiments, each of third probes 530 has at least one common oligonucleotide sequence. That is, among the different types of first probes 510 and second probes 520, the secondary regions 514 among the different types of first probes 510 can have a first common oligonucleotide sequence such that all third probes 530 hybridize to the first common oligonucleotide sequence. In some embodiments, among the different types of second probes 520, the secondary regions 524 can have a second common oligonucleotide sequence such that all third probes 530 hybridize to the second common oligonucleotide sequence.

Where both secondary regions 514 and 524 have common oligonucleotide sequences among the different types of first and second probes 510 and 520, respectively, the second composition contains only a single type of third probe 530. Where only one of secondary regions 514 and 524 has a common oligonucleotide sequence among the different types of first and second probes 510 and 520, or neither of the secondary regions has a common oligonucleotide sequence, the second composition contains multiple pluralities of third probes 530, where each plurality of third probes 530 is a different type of third probe 530 that selectively hybridizes to a particular combination of a first probe 510 and a second probe 520.

Following exposure of the sample to the second composition, the sample is exposed to a third composition that contains multiple pluralities of fourth probes 540. In some embodiments, each plurality of fourth probes 540 is a different type of fourth probe that selectively hybridizes to a particular combination of a first probe 510 (i.e., to a specific oligonucleotide sequence of secondary region 514) and a second probe 520 (i.e., to a specific oligonucleotide sequence of secondary region 524). In this manner, each different type of reporter moiety 106 that is introduced via the fourth probes 540 is selectively associated with only one combination of a type of first probe 510 and a type of second probe 520, and therefore, only one type of RNA 20.

Alternatively, in certain embodiments, one or more of the pluralities of fourth probes 540 hybridizes to more than one combination of a first probe 510 and a second probe 520. In particular, different combinations of first probe 510 and second probe 520 can be selected to hybridize to different portions of the same RNA 20. A single type of fourth probe 540 can hybridize to each combination of first probe 510 and second probe 520 that hybridizes to a different portion of a type of RNA 20. The third composition can include multiple types of fourth probes 540, each of which hybridizes to multiple combinations of first probe 510 and second probe 520 that hybridize to different portions of a type of RNA 20.

In certain embodiments, fourth probes 540 can be introduced prior to third probes 530. For example, following exposure of the sample to the first composition, the sample can be exposed to a second composition that includes multiple pluralities of fourth probes 540 as discussed above. The sample can then be exposed to a third composition that includes third probes 530. The fourth probes 540 and third probes 530 can be distributed among the second and third compositions, respectively, as discussed above.

In some embodiments, the sample can be exposed to the third probes 530 and fourth probes 540 in a single composition. That is, following exposure of the sample to the first composition, the sample can be exposed to a second composition that includes third probes 530 and multiple pluralities of fourth probes 540. The third and fourth probes can be distributed in the second composition as discussed above.

Exposure of the sample 10 to pluralities of probes according to any of the workflows described herein yields a sample in which different RNAs are labeled with different probes (or combinations of probes, where each combination is itself a “probe” formed by multiple oligonucleotides). Each probe includes a capture moiety 102 and a detection moiety 104, and each detection moiety 104 includes one or more reporter moieties 106 that are selectively associated with the particular type of RNA 20 to which capture moiety 102 binds. Samples in which one or more types of RNAs have been labeled with probes can then be analyzed to determine the presence of the one or more types of RNAs in the sample and their spatial locations.

### Detection of Labeled RNA Species

With one or more types of RNAs 20 in sample 10 bound to a different types of detection moieties 104 through different capture moieties 102, the RNAs in the sample can be detected and quantified through successive cycles of optical label binding and imaging in the sample. For RNAs bound to detection moieties 104 with reporter moieties 106 that include oligonucleotide label regions, optical labels that include an oligonucleotide conjugated to a species that generates an optical signal can be hybridized to the oligonucleotide label regions of the reporter moieties. By measuring the optical signals generated by each optical label, individual RNAs in the sample can be identified and quantified, as each different type of RNA in the sample is associated with a different type of detection moiety 104 that includes reporter moieties featuring a different combination of label regions. As such, the combination of optical signals attributable to the optical labels that hybridize to the reporter moieties of each type of detection moiety is unique.

Where the label regions of reporter moieties 106 are oligonucleotide-based, the optical labels that are used in the methods described herein generally also include an oligonucleotide. The oligonucleotides of the optical labels each have a sequence that is at least partially, or fully, complementary to one of the label regions of the reporter moieties 106 of the detection moieties 104 conjugated to the RNAs in the sample. Oligonucleotides of the optical labels can each have the same or a different number of nucleotides. More generally, the oligonucleotides of the optical labels can have the same properties as the oligonucleotides of the label regions discussed above.

In general, each optical label includes at least one species that generates an optical signal, which is referred to herein as a “dye.” That is, a dye is a moiety that interacts with incident light, and from which emitted light can be measured and used to detect the presence of the dye in a sample. In general, a dye can be a fluorescent moiety, an absorptive moiety (e.g., a chromogenic moiety), or another type of moiety that emits light, or modifies incident light passing through or reflected from a sample where the dye is present so that the presence of the dye can be determined by measuring changes in transmitted or reflected light from the sample.

In certain embodiments, the optical label can include a hapten. The hapten can subsequently (or concurrently) be bound to a dye to provide a labeling moiety that can be detected by measuring emitted, transmitted, or reflected light from the sample.

A wide variety of different dyes can generally be used in optical labels. For example, the dye can be a xanthene-based dye, such as a fluorescein dye and/or a rhodamine dye. Examples of suitable fluorescein and rhodamine dyes include, but are not limited to, fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′, 5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110.

The dye can also be a cyanine-based dye. Suitable examples of such dyes include, but are not limited to, the dyes Cy3, Cy5 and Cy7. The dye can also be a coumarin dye (e.g., umbelliferone), a benzimide dye (e.g., any of the Hoechst dyes such as Hoechst 33258), a phenanthridine dye (e.g., Texas Red), an ethidium dyes, an acridine dyes, a carbazole dye, a phenoxazine dye, a porphyrin dye, a polymethine dye (e.g., any of the BODIPY dyes), and a quinoline dye.

When the dye is a fluorescent species, the dye can be a moiety corresponding to any of the following non-limiting examples and/or derivatives thereof: pyrenes, coumarins, diethylaminocoumarins, FAM, fluorescein chlorotriazinyl, fluorescein, R1 10, JOE, R6G, tetramethylrhodamine, TAMRA, lissamine, napthofluorescein, Texas Red, Cy3, and Cy5.

In certain embodiments, the dye can include one or more quantum dot-based species. Quantum dot-based fluorophores are available with fluorescence emission spectra in many different spectral bands, and suitable quantum dot-based dyes can be used as labeling species in the methods described herein.

### Multiple-Cycle Detection of RNAs

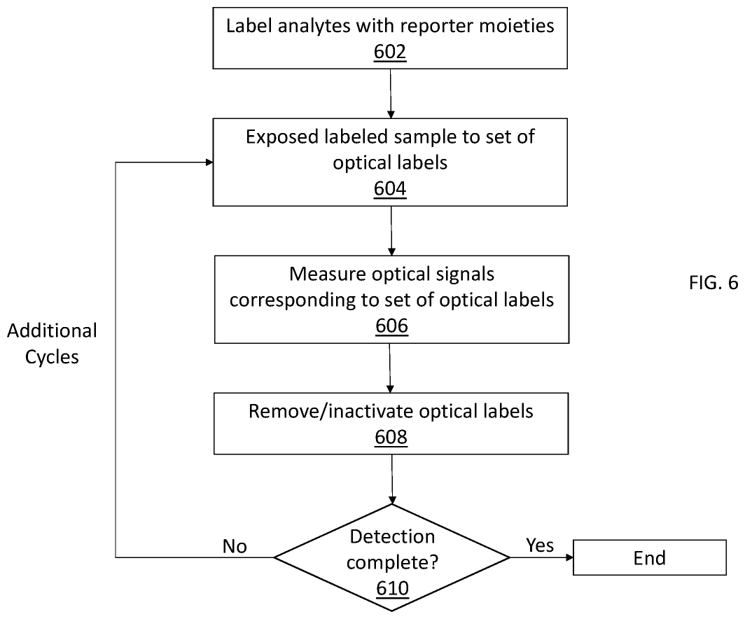

To detect RNAs in a sample, one or more cycles of optical label hybridization and detection are generally performed. FIG. 6 is a flow chart that shows a set of example steps for detecting and quantifying multiple types of RNAs in a sample. In step 602, the multiple RNAs are conjugated to detection moieties, with each different type of RNA conjugated to a different type of detection moiety having one or more reporter moieties, each with a unique combination of label regions as discussed above.

Next, in step 604, the sample is exposed to a set of one or more optical labels. The set of optical labels typically includes between 1 and 8 different optical labels (e.g., two, three, or four different optical labels), but can generally include any number of optical labels in each cycle of the flow chart of FIG. 6.

In general, one or more of the optical labels introduced in step 604 can hybridize with one or more of the label regions of the different types of reporter moieties in the detection moieties in the sample. To increase the efficiency with which different types of reporter moieties are identified (e.g., by reducing the number of detection cycles), the set of optical labels can be selected such that, for at least one (and more generally, more than one) of the different types of reporter moieties present in the sample, multiple different optical labels of the introduced set hybridize to different label regions of the reporter moiety/moieties, and generate optical signals. In this manner, multiple label regions of these reporter moieties can be identified in a single detection cycle, reducing the number of cycles required to fully elucidate all of the label regions associated with the reporter moieties. By selecting the optical label set in each cycle such that multiple different optical labels hybridize to different label regions of one or more of the different reporter moieties present in the sample, the number of detection cycles can be more efficiently utilized to identify the different reporter moieties, and therefore, to detect specific RNAs in the sample.

Conversely, as a simplification of the above general optical label selection and utilization methodology, in some embodiments, the set of optical labels can optionally be selected such that in step 604, for each different type of RNA conjugated to a different type of detection moiety (where each different type of detection moiety includes a different type of reporter moiety), at most one of the optical labels in the set hybridizes to each different type of reporter moiety. In other words, in each detection cycle that includes step 604, at most one of the label regions of each different type of reporter moiety can be identified. As a consequence of this constraint on the selection of optical labels, each type of reporter moiety that includes D different label regions can be fully identified following a minimum of D detection cycles. As the number of different types of RNAs increases, the number of detection cycles that are performed to fully identify each of the different types of reporter moieties generally increases, particularly if the number of different optical labels in each set remains constant.

While this choice of the optical label set may be less efficient than the more general methodology in which more than one optical label hybridizes to one or more of the different types of reporter moieties in the sample as described above, this choice of optical label set can be useful in some circumstances. For example, in samples where optical labels may interact with one another (e.g., by fluorescence resonance excitation transfer, donor-acceptor quenching, or photoinduced electron transfer), it may be disadvantageous to introduce certain optical labels in a manner such that they hybridize to a common reporter moiety, as positioning the optical labels in such close proximity may promote such interactions, which disrupt successful detection of the label regions to which they hybridize by extinguishing, masking, or otherwise interfering with the signals generated by the optical labels. In such circumstances, by selecting the set of optical labels so that at most one optical label from the set hybridizes to each different type of reporter moiety in the sample, interactions between different types of optical labels can be reduced.

Next, in step 606, optical signals corresponding to the optical labels of the set are measured. In some embodiments, the optical signals are measured by obtaining one or more images (e.g., multispectral images) of the sample with the set of optical labels hybridized to complementary label regions of different reporter moieties associated with different RNAs of the sample. To obtain the one or more images, the sample is exposed to incident light, and signal radiation generated by the optical labels (e.g., fluorescence emission) is detected using an imaging detector such as a CCD array or CMOS-based array detector.

In general, each of the different optical labels that is introduced in step 604 generates signal radiation according to a different spectral distribution, and is therefore associated with a different detection channel. In practice, signal radiation in different detection channels can be detected in a variety of ways. In some embodiments, where each detection channel is well separated spectrally from the other detection channels, the signal radiation generated by each different type of optical label is relatively well isolated spectrally in a distinct detection channel. As such, signal radiation attributable to each of the different types of optical labels can readily be isolated and detected by spectral filtering (e.g., with a plurality of optical bandpass filters) and/or by using a spectrally resolving detector, such as a grating, prism, or other spectrally dispersive element in conjunction with a CCD array or CMOS-based array detector.

In certain embodiments, the spectral distributions of signal radiation generated by the different optical labels may overlap to a degree that is not insignificant, such that optical filtering and spectral dispersion methods alone are insufficient to isolate signal radiation generated by each of the different optical labels. Because the spectral distributions of the signal radiation are spectrally convolved to some extent, accurate detection of signals generated by each of the optical labels may therefore involve more complex spectral deconvolution techniques to accurately separate and assign measured signals to specific optical labels.

In such circumstances, sample images that include signal radiation from multiple different optical labels can optionally be decomposed into a set of images, in which each image in the set corresponds substantially only to signal radiation from one optical label. A variety of methods can be used to perform such decompositions, including for example spectral unmixing methods that involve performing an eigenvector decomposition of the measured optical signals into individual contributions from “pure” spectral components (e.g., contributions from each optical label).

In general, RNAs are present in the sample at different spatial locations. As such, for each pixel in an image of the sample, any optical signals measured at that pixel will arise from optical labels hybridized to a reporter moiety that is part of a detection moiety linked to a capture moiety specifically bound to one RNA at a location in the sample corresponding to the pixel. In embodiments where, in a detection cycle, multiple optical labels hybridize to a reporter moiety, multiple optical signals are therefore generated and detected at a pixel corresponding to the reporter moiety. As described above, if the multiple optical signals are well separated spectrally, they can be resolved and detected by spectral filtering methods. Alternatively, if the multiple optical signals overlap spectrally, the measured signal at the pixel—which corresponds to a convolution of the multiple signals—can be decomposed into component signals (e.g., component images) that individually correspond to an optical signal generated substantially only by a single one of the optical labels.

Further, as described above, if the set of optical labels is selected so that, at most, one optical label in the set hybridizes to each reporter moiety 106 in the sample, then each pixel in the set of one or more images will include, at most, an optical signal corresponding to one of the optical labels in the set. In such circumstances, methods for spectrally decomposing the measured signals are generally not required to separate signals generated by multiple optical labels. However, such methods can still be used, for example, to compensate for the effects of autofluorescence and spectral contributions that may arise from other species present in the same that are not of interest.

It should also be noted that in each image of the sample, certain pixels may be “dark” (i.e., have zero or very low aggregate signal intensity). A dark pixel indicates that none of the optical labels hybridized to reporter moieties at a location in the sample corresponding to the dark pixel during the current detection cycle. If no optical signals are measured at a particular pixel among all images through all detection cycles, then none of the different types of RNAs targeted by the capture moieties was present at the location in the sample corresponding to the dark pixel.

Step 606 yields a set of one or more images of the sample. Particular pixels at a common location in the set of images correspond to the same location in the sample, which is represented by the common pixel location in the images. Collectively, pixels across the set of images that correspond to a common pixel location are associated with optical signals generated by optical labels that hybridize at the corresponding location in the sample. Because the optical signals generated by each different type of optical label in a detection cycle are known, the presence of particular label regions at each pixel location in the images can be determined. After all detection cycles are complete, the particular set of label regions present at each pixel location can be used to determine the type of reporter moiety 106 present at the pixel location, and therefore, the RNA present at the pixel location.

Following step 606, the set of optical labels can be removed from the sample or inactivated in step 608. A variety of different methods can be used in step 608 for optical label removal or inactivation.

In some embodiments, optical labels that hybridize to label regions of reporter moieties can be readily removed by dehybridization. Dehybridization can be accomplished using various methods including, but not limited to: exposure to one or more chaotropic reagents; thermally-induced dehybridization via heating; toehold mediated strand displacement (TMSD); and enzymatic strand displacement using enzymes such as RNAse, DNAse.

In certain embodiments, portions of the optical labels can be removed from the reporter moieties, while other portions of the optical labels remain in the sample. For example, one or more reducing agents can be used to cleave covalent bonds that link a dye moiety to an oligonucleotide in an optical label. The cleaved dye moieties can then be washed from the sample. The oligonucleotides can optionally remain hybridized to label regions in the reporter moiety. A variety of different reducing agents can be used for this purpose. For example, tri(2-carboxyethyl)phosphine (TCEP) can be used to cleave dye moieties that are linked via disulfide bonds to oligonucleotides in optical labels.

In some embodiments, optical labels are not removed from the sample, but are instead inactivated so that they do not generate optical signals in subsequent detection cycles. Various methods can be used for inactivation of optical labels. For example, in certain embodiments, chemical bleaching of optical labels can be used to inactivate the labels.

Next, in step 610, if analysis of the RNAs present in the sample is complete, then the workflow ends. However, if analysis is not complete, one or more additional cycles of steps 604, 606, and 608 are performed. In each additional cycle, a set of optical labels are introduced into the sample and associate with label regions of the reporter moieties in the sample. Optical signals corresponding to the set of optical labels are measured and optionally decomposed as described above, before the optical labels are optionally removed from the sample and/or deactivated.

The workflow shown in FIG. 6 can be repeated for any number of cycles to detect and quantify RNAs in the sample. In some embodiments, each set of optical labels that is used in a particular cycle is unique; that is, none of the optical labels are re-used in subsequent cycles. In this manner, individual label regions of each reporter moiety are detected only once through the entire workflow.

In certain embodiments, to perform error checking and/or to correct for binding and imaging errors such as incomplete hybridization and/or optical imaging aberrations, certain optical labels can be applied to the sample in more than one cycle in FIG. 6. By doing so, for example, optical signals measured in prior cycles can be verified, and optical signals that may have been absent in prior cycles (e.g., due to incomplete hybridization of the optical labels) can be measured in a later cycle.

It should also be noted that while in some embodiments each of the optical labels is introduced into the sample only once during the workflow shown in FIG. 6, optical labels that bind to different label regions of reporter moieties, but have the same dye, can be used in different cycles of the workflow. That is, provided that different optical labels in the same cycle do not include the same dye, measured signals can be unambiguously attributed to the different optical labels. This unambiguous assignment remains possible when different optical labels include the same dye, provided the optical labels are applied and corresponding signals measured during different cycles in FIG. 6.

In general, the workflow in FIG. 6 typically continues until a sufficient number of cycles have been performed to unambiguously identify each of the RNAs in the sample. In some embodiments, the workflow continues until optical labels that bind or associate with each of the label regions of each of the reporter moieties 106 have been introduced, and signals attributable thereto have been measured.

In certain embodiments, depending upon the number of RNAs, the number of different reporter moieties, and the number of label regions in each reporter moiety, it may be possible to unambiguously identify each of the RNAs without introducing and measuring optical labels that bind or associate with every label region of every type of reporter moiety. For example, RNAs in the sample can be conjugated to detection moieties that include reporter moieties with 6 label regions, but it may be possible to unambiguously identify certain RNAs of interest by binding or associating optical labels to only a subset (e.g., 3 or 4) of the label regions of some or all of the reporter moieties. In this manner, one or more detection cycles may be omitted, saving the expense and time associated with the omitted cycles. This method may be applied, for example, to assays in which a limited number of RNAs are of interest, while the generalized assay permits identification of a much larger number of different RNAs. Foregoing detection cycles that only provide information about RNAs that are not of interest, or that do not provide additional information that is useful for identifying RNAs that are of interest, shortens the time associated with the assay, and may reduce the associated cost by limiting the number of different optical labels that are used.

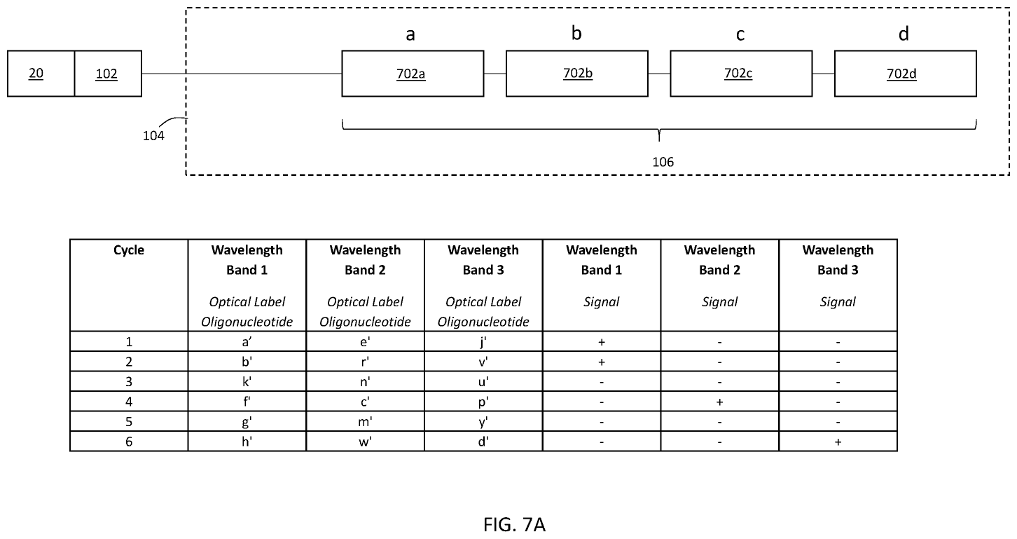

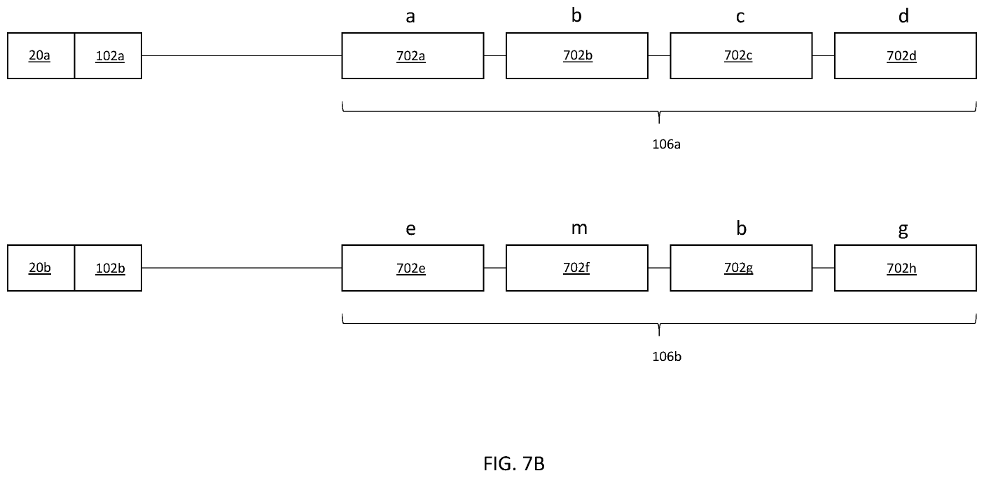

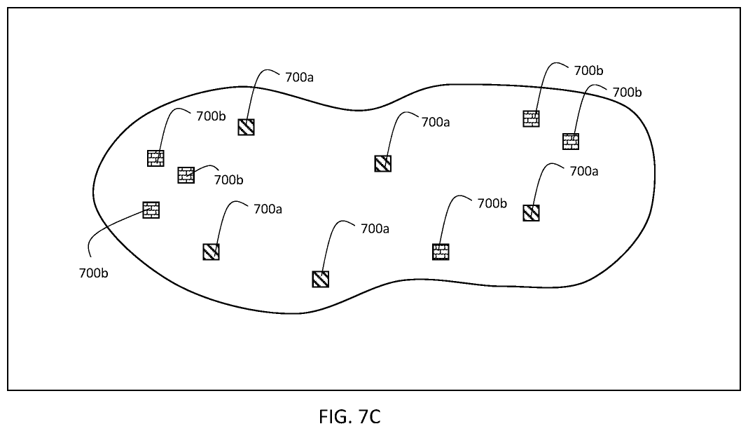

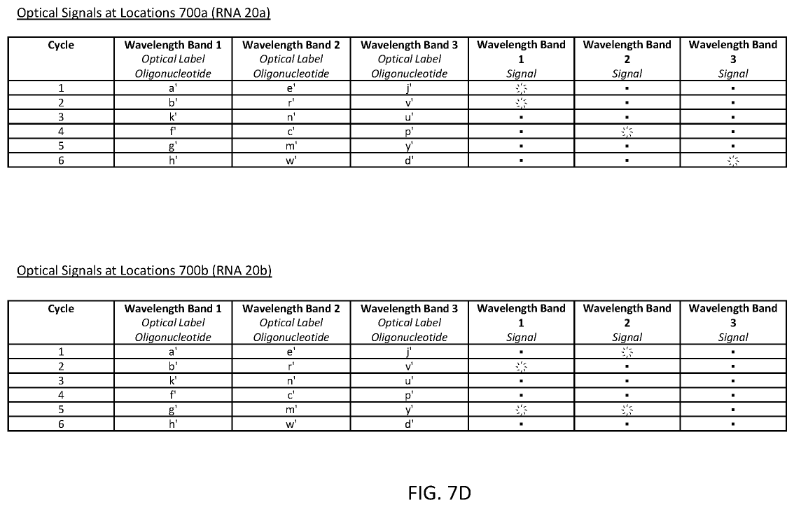

FIG. 7A is a schematic diagram showing an example illustrating the detection of a RNA 20 in sample 10 via multiple cycles of hybridization and dehybridization of oligonucleotide-based optical labels. RNA 20 is bound to capture moiety 102, which is conjugated to a detection moiety 104 that includes one or more reporter moieties 106. Each of the reporter moieties 106 includes 702a-d. Each one of label regions 702a-d includes a distinct oligonucleotide sequence, and the respective sequences are labeled a, b, c, and d for reference. For an oligonucleotide sequence designated x, a partially or fully complementary sequence that hybridizes to sequence x is designated x′. Thus, for example, sequence a′ is complementary to and hybridizes to sequence a, sequence b′ is complementary to and hybridizes to sequence b, and so forth.

To detect RNA 20, multiple detection cycles are performed as described above in connection with FIG. 6. In each cycle, one or more optical labels with oligonucleotide sequences are introduced into the sample. Optical labels with sequences that are at least partially complementary to sequences of label regions 702a-d hybridize to the corresponding label regions. Measurement of the signals generated by the hybridized optical labels reveals the presence of the label region at particular spatial locations in the sample. Co-location in the sample of signals attributable to each of the label regions in a particular reporter moiety reveals the presence the reporter moiety at that location in the sample, and therefore, the presence of RNA 20 to which the reporter moiety is conjugated.

In general, within each detection cycle, each different type of optical label includes a different oligonucleotide sequence, and generates a distinguishable optical signal. As such, within each cycle, following hybridization of one of more of the optical labels to complementary label regions of reporter moieties, the optical signals corresponding to the different types of optical labels can be measured and distinguished, identifying the different types of optical labels that have hybridized to complementary label regions of reporter moieties.

Optical signals generated by the different types of optical labels can be distinguishable in various ways. In some embodiments, for example, different types of optical labels include different dyes (e.g., fluorescent dyes) that emit light in different wavelength bands (i.e., bands that have different central wavelengths and/or different wavelengths of maximum emission intensity). If the wavelength bands are sufficiently separated spectrally, the optical signals can be distinguished from one another by spectral filtering and/or related techniques.

For optical signals that are not as well separated spectrally (e.g., when relatively higher numbers of different types of optical labels are used in a detection cycle), or that have non-Gaussian emission band shapes, a variety of computational techniques can be used to separate measured optical signals into component signals that individually correspond substantially to only one of the optical labels. For example, eigenvector decomposition methods such as spectral unmixing can be used to decompose multispectral images of a sample into a set of single-component images, each corresponding to spectral contributions from only one of the different types of optical labels.

To detect RNA 20 in sample 10, individual label regions of each reporter moiety 106 in FIG. 7A are detected in different cycles of the detection sequence. For purposes of illustration, within each detection cycle in FIG. 7A, three different types of optical labels are introduced.

Each of the different types of optical labels includes a different oligonucleotide, and a fluorescent moiety that generates an optical signal in one of three different wavelength bands. Optical signals in each of the three different wavelength bands are distinguishable from one another using the methods discussed above.

It should be noted that the three different wavelength bands can be the same or different in different detection cycles. In some embodiments, for example, three different types of optical labels are introduced in each detection cycle, and each of the different types of optical labels generates an optical signal in only one of three different wavelength bands, with the three different wavelength bands remaining the same among all cycles. In other words, the same set of three different wavelength bands is re-used in each detection cycle.

In certain embodiments, some or all of the wavelength bands in which the different types of optical labels generate optical signals are different from one cycle to the next. That is, some of wavelength bands may be re-used from one cycle to the next, or none of the wavelength bands may be re-used. In general, there are no constraints on the manner in which different wavelength bands can be re-used or not re-used, except that within a particular detection cycle, each of the different types of optical labels generates an optical signal that is distinguishable from optical cycles generated by other types of optical labels within the cycle.

It should also be used that the introduction of three different types of optical labels in each detection cycle in FIG. 7A is merely an example for illustrative purposes. More generally, any number of different types of optical labels can be introduced in each cycle. The number of different types of optical labels that can be introduced can be the same among all cycles, or can vary among the cycles. Further, in some embodiments, each type of optical label is introduced only once among all detection cycles, as shown in FIG. 7A. More generally, however, some or all of the optical labels can be introduced in more than one (e.g., two or more, three or more, four or more, five or more, or even more) of the detection cycles. Introducing one or more of the optical labels in multiple detection cycles can allow verification of previously-detected optical signals that have been attributed to specific optical labels, for example.

The table in FIG. 7A shows an example of several cycles of optical label hybridization and measurement to detect RNA 20 in the sample. In each cycle, a first type of optical label generates an optical signal in Wavelength Band 1, a second type of optical label generates an optical signal in Wavelength Band 2, and a third type of optical label generates an optical signal in Wavelength Band 3. It should be noted that Wavelength Bands 1, 2, and 3 may be the same from one cycle to the next, or some or all of the Wavelength Bands may differ between any two cycles. The indicators “1”, “2”, and “3” designate only that the Wavelength Bands are different, and optical signals generated in each Wavelength Bands are distinguishable to allow identification of the optical labels that generate the signals, and therefore, the complementary label regions to which they are hybridized.

In cycle 1, optical labels with respective sequences a′, e′, and j′ are introduced into the sample. The first type of optical label with sequence a′ generates an optical signal in Wavelength Band 1, the second type of optical label with sequence e′ generates an optical signal in Wavelength Band 2, and the third type of optical label with sequence j′ generates an optical signal in Wavelength Band 3. Only the optical label with sequence a′ hybridizes to reporter moiety 106 (i.e., to label region 702a with sequence a). Thus, in cycle 1, a positive signal (i.e., a non-zero optical signal) is measured at the location of RNA 20 in the sample in Wavelength Band 1. No positive signals are measured in either Wavelength Band 2 or Wavelength Band 3.

In cycle 2, optical labels with respective sequences b′, r′, and v′ are introduced. The first type of optical label with sequence b′ generates an optical signal in Wavelength Band 1, the second type of optical label with sequence r′ generates an optical signal in Wavelength Band 2, and the third type of optical label with sequence v′ generates an optical signal in Wavelength Band 3. Only the optical label with sequence b′ hybridizes to reporter moiety 106 (to label region 702b with sequence b). Therefore, in cycle 2, a positive signal is measured at the location of RNA 20 in the sample in Wavelength Band 1. No positive signals are measured in either Wavelength Band 2 or Wavelength Band 3.

The different types of optical labels introduced in cycles 3-6 are shown in FIG. 7A as well. Positive signals are measured at the location of RNA 20 in cycles 4 (in Wavelength Band 2 but not in Wavelength Bands 1 or 3) and 6 (in Wavelength Band 3 but not in Wavelength Bands 1 or 2). No positive signals are measured in any of the Wavelength Bands in cycles 3 or 5, as none of the optical labels in those cycles hybridizes to any of the label regions of reporter moiety 106. The combination of positive signals measured in cycles 1 (in Wavelength Band 1), 2 (in Wavelength Band 1), 4 (in Wavelength Band 2), and 6 (in Wavelength Band 3), directly identifies that sequences a, b, c, and d are present in reporter moiety 106, as those particular Wavelength Bands are assigned to optical labels with complementary oligonucleotide sequences a′, b′, c′ and d′ in cycles 1, 2, 4, and 6, respectively. As the combination of label regions a-b-c-d is specific for a capture moiety 102 that targets RNA 20, the presence of RNA 20 can be unambiguously determined in the sample. Further, the measured signal intensity is correlated with the amount of RNA 20 at each location in the sample, and therefore RNA 20 can be spatially quantified in the sample.

It should also be noted that the foregoing methods do not distinguish among different orderings of label regions within reporter moiety 106. In the example of FIG. 7A, by performing the 6 detection cycles shown, it can be determined that reporter moiety 106 includes label regions a, b, c, d. However, the specific order of label regions within reporter moiety 106 is not determined. Thus, the specific order of the label regions could be a-b-c-d, b-d-c-a, c-b-d-a, d-b-a-c, or any other ordered, non-repeating combination of the sequences a, b, c, and d.

In the example of FIG. 7A, in each set of optical labels that is introduced in each of the 6 detection cycles, at most one of the optical labels hybridizes to a label region of reporter moiety 106. However, as discussed above, more generally the sets of optical labels are selected such that in one or more of the detection cycles, multiple different optical labels can hybridize to individual reporter moieties 106, and generate optical signals that are detected and used to identify corresponding label regions in the reporter moieties. By selecting the sets of optical labels in this manner, the overall efficiency of the assay can be increased by making increased use of the available detection channels during each detection cycle.

The example shown in FIG. 7A is representative, and illustrates the detection of a single RNA 20. In general, however, a sample includes many different RNAs, and the methods, compositions, and kits described herein are used to detect many different RNAs. For example, in some embodiments, 10 or more (e.g., 20 or more, 30 or more, 40 or more, 50 or more, 60 or more, 70 or more, 80 or more, 100 or more, 120 or more, 140 or more, 160 or more, 200 or more, 300 or more, 400 or more, 500 or more, 700 or more, 1000 or more, 1500 or more, 2000 or more, 3000 or more, 5000 or more, 7000 or more, 10000 or more, 15000 or more, 20000 or more, 30000 or more, 40000 or more, 50000 or more, or even more) different RNAs are detected in a sample using the methods, compositions, and/or kits described herein.

When a sample contains multiple different RNAs, each different type of RNA selectively binds to a different type of probe or combination of probes that include(s) a capture moiety specific to that type of RNA, and a detection moiety linked to the capture moiety that includes one or more reporter moieties specifically associated with that type of RNA. The multi-cycle detection methods described herein are used to detect the multiple different types of RNAs. During each detection cycle, the different optical labels introduced in that cycle can hybridize to complementary label regions in the reporter moieties in the sample that are associated with different types of RNAs. In general, in each cycle, the set of optical labels that are introduced can be selected so that multiple optical labels hybridize to one or more of the different types of reporter moieties, which ensures that individual detection cycles are efficiently used to elucidate the label regions—and therefore the reporter moieties—that are present in each location of the sample.

FIG. 7B shows two probes that specifically bind to two different RNAs in sample 10. Specifically, the first probe includes a capture moiety 102a that specifically binds to a first type of RNA 20a, and is linked to a detection moiety that includes one or more reporter moieties 106a, each of which includes label regions 702a-702d with oligonucleotide sequences a, b, c, and d, respectively. The second probe includes a capture moiety 102b that specifically binds to a second type of RNA 20b, and is linked to a detection moiety that includes one or more reporter moieties 106b, each of which includes label regions 702e-702h with oligonucleotide sequences e, m, b, and g, respectively.

FIG. 7C is a schematic image of sample 10. In sample 10, RNA 20a is present at locations 700a, while RNA 20b is present at locations 700b. It should be noted that only one type of RNA is present at each physical location in sample 10. In other words, two RNA molecules in sample 10 are not spatially coincident anywhere in sample 10. Instead, each RNA molecule is present at a distinct location, although particular pairs of RNA molecules may be very closely spaced within sample 10. The methods described herein can be used no matter how closely spaced two RNA molecules are in the sample.

FIG. 7D shows the measured optical signals from sample 10 at locations 700a and 700b over 6 detection cycles in tabular form. The upper table shows the measured optical signals at locations 700a (where RNA 20a is located in the sample). The lower table shows the measured optical signals at locations 700b (where RNA 20b is located in the sample). In each table, the sunburst character indicates that an optical signal was measured in a particular wavelength band, and the solid square character indicates that no optical signal was measured in a particular wavelength band.

As shown in column 1 of each table, 6 detection cycles were performed. Three optical labels were introduced in each detection cycle. The oligonucleotide sequences of each of the optical labels in each detection cycle, and the wavelength bands in which they generate optical signals, are shown in columns 2-4 of each table. Because the detection cycles were performed on the whole sample 10, columns 1-4 are identical in both tables.

The upper table indicates that positive optical signals were measured at locations (e.g., pixels) 700a in sample 10 in the following ordered pairs: (cycle 1, wavelength band 1), which corresponds to an optical label with oligonucleotide sequence a′; (cycle 2, wavelength band 1), which corresponds to an optical label with oligonucleotide sequence b′; (cycle 4, wavelength band 2), which corresponds to an optical label with oligonucleotide sequence c′; and (cycle 6, wavelength band 3), which corresponds to an optical label with oligonucleotide sequence d′. These positive optical signals are measured because reporter moiety 106a, which is exclusively present at locations 700a in the sample, includes label regions with oligonucleotide sequences a, b, c, and d.

The lower table indicates positive signals that were measured at locations (e.g., pixels) 700b in sample 10. Reporter moiety 106b is exclusively present at locations 700b, and contains label regions with oligonucleotide sequences e, m, b, and g. The positive signals measured at locations 700b in the sample are represented by the following ordered pairs: (cycle 1, wavelength band 2), which corresponds to an optical label with oligonucleotide sequence e′; (cycle 2, wavelength band 1), which corresponds to an optical label with oligonucleotide sequence b′; (cycle 5, wavelength band 1), which corresponds to an optical label with oligonucleotide sequence g′; and (cycle 5, wavelength band 2), which corresponds to an optical label with oligonucleotide sequence m′.

Following the 6 detection cycles shown in FIG. 7D, each of the image pixels corresponding to sample locations 700a is associated with positive signals for label regions a, b, c, and d. Because only reporter moiety 106a corresponds to that combination of label regions, the presence of RNA 20a at locations 700a can be unambiguously identified. Similarly, each of the image pixels corresponding to sample locations 700b is associated with positive signals for label regions e, b, g, and m. Only reporter moiety 106b corresponds to that combination of label regions, and so the presence of RNA 20b at locations 700b can be unambiguously identified.

As is apparent from the results of cycle 2, when different reporter moieties contain the same label region (reporter moieties 106a and 106b each contain a label region with oligonucleotide sequence b in FIG. 7B), optical signals are measured from optical labels that hybridize to each of the different reporter moieties in a detection cycle in which the complementary optical label (e.g., an optical label with oligonucleotide sequence b′) is introduced. However, the reporter moieties are associated with different RNAs due to the specific nature of the interaction between the capture moieties and the RNAs. In the present example, reporter moiety 106a is associated only with RNA 20a by virtue of the specific interaction between capture moiety 102a and RNA 20a, and reporter moiety 106b is associated only with RNA 20b by virtue of the specific interaction between capture moiety 102b and RNA 20b. As a result, the optical signals corresponding to reporter moiety 106a will be measured only at locations 700a, and the optical signals corresponding to reporter moiety 106b will be measured only at locations 700b, even though the optical signals are generated by the same optical label with oligonucleotide sequence b′.

Because RNAs 20a and 20b are at spatially distinct locations in sample 10, the measured optical signals corresponding to the optical label with sequence b′ have different meanings. Signals measured at locations 700a correspond to reporter moiety 106a and RNA 20a, while signals measured at locations 700b correspond to reporter moiety 106b and RNA 20b.

In general, it is not known a priori where any of the RNAs are located within the sample. Instead, the locations of particular types of RNAs are determined from the accumulated optical signal measurements at each pixel location. Specifically, each location in a sample (which corresponds to a particular pixel in the sample images that are obtained for detection of optical signals) is associated with a set of measured optical signals generated by optical labels during the multiple cycles of detection. At each location/pixel, the set of measured optical signals is associated with certain optical labels that were introduced during the detection cycles. Because the oligonucleotide sequences of the optical labels were known, the set of measured optical signals at each location/pixel is associated with a set of label regions at that location/pixel.

Each different type of reporter moiety contains a unique combination of label regions, without regard to ordering of the label regions. Consequently, the set of label regions at a particular location/pixel uniquely associates that location/pixel with a particular reporter moiety, and therefore, a particular RNA at that location/pixel. In this manner, at each location/pixel within the sample, the presence of any one of the RNAs can be detected. Referring again to the example in FIGS. 7B-7D above, the co-location of label regions a, b, c, and d at locations 700a in sample 10 identifies RNA 20a at locations 700a. Similarly, the co-location of label regions e, m, b, and g at locations 700b in sample 10 identifies RNA 20b at locations 700b.

For locations/pixels at which no set of label regions can be determined (i.e., no optical signals are measured), no RNA is located at those locations/pixels. For locations/pixels at which an incomplete or erroneous set of label regions is determined, the presence or absence of an RNA and the nature of the RNA at those locations/pixels is indeterminate if the determined set of label regions cannot be corrected.

As discussed previously, in some embodiments, it can be advantageous to introduce one or more optical labels in two or more of the detection cycles in a multi-cycle detection procedure. For example, referring again to the example of FIG. 7A, after cycle 6, an additional detection cycle 7 can be performed in which one or more of the optical labels previously introduced is introduced again. By way of example only, consider a detection cycle in which optical labels with oligonucleotide sequences a′ (associated with wavelength band 1), s′ (associated with wavelength band 2), and q′ (associated with wavelength band 3) are introduced. The optical labels corresponding to sequences s′ and q′ will not hybridize to reporter moiety 106, and therefore generate no measured signal in any of the wavelength bands. The optical labels corresponding to sequence a′ will hybridize again to reporter moiety 106, and generate optical signals in wavelength band 1.

The signals are detected, as described above, by obtaining one or more images of the sample. Nominally, the spatial distribution of signals corresponding to the optical label with sequence a′ should be the same as the spatial distribution of signals corresponding to the optical label with sequence a′ in cycle 1. Accordingly, the measured optical signals generated by the optical labels with sequence a′ at each of the locations/pixels in cycle 7 can be used as a verification of the optical signals corresponding to optical labels with sequence a′ that were measured in cycle 1. Discrepancies may be corrected, or pixels at which discrepancies are observed can be marked and omitted from further analysis.

Thus, while introducing optical labels in more than one detection cycle can increase the time over which the assay is performed, doing so can also provide verification of previously measured optical signals, which can be an important consideration when the sample is of poor quality and/or certain RNAs are not easily or reproducibly bound to capture moieties. It should be noted that the introduction of optical labels in more than one detection cycle is advantageous when optical labels are dehybridized following measurement of optical signals. If the optical labels are instead deactivated or only partially removed, later re-introduction of the same optical labels may be more difficult.

### Calibration of Detected Optical Signals

In some embodiments, additional calibration and error checking steps can be performed during multi-cycle hybridization and detection of optical labels. For example, referring again to FIGS. 6 and 7A-7D, to identify a reporter moiety, optical labels that hybridize to complementary label regions of the reporter moiety are typically introduced once during a multi-cycle hybridization and detection procedure. A single exposure of all reporter moieties in a sample to a particular optical label is generally sufficient to ensure that all complementary label regions among all reporter moieties in the sample hybridize to the particular optical label, and optical signals generated by the optical label are detected.

However, in some embodiments, one or more of the optical labels are hybridized to reporter moieties in more than one cycle of the multi-cycle detection procedure. Repeating the exposure to one or more optical labels can be performed as a verification of measured detection signals arising from the optical labels. For example, an initial hybridization of a particular optical label to reporter moieties in a sample yields a spatial distribution of optical signals corresponding to the optical label. By repeating exposure to the same optical label in a later cycle, the measured spatial distribution of optical signals (i.e., obtained from an image of the sample) can be verified and, if necessary, corrected based on the second measured spatial distribution of optical signals obtained after the repeated exposure. Verification can be used both to ensure that spurious optical signals were not measured initially, and that optical signals that should have been measured initially were not undetected. By verifying the measured optical signals in such a manner, measurements of the spatial distributions of particular RNAa can be confirmed. Furthermore, quantitative measurements of particular RNAs, which are obtained by integrating spatial distributions of the RNAs, can also be verified.

In certain embodiments, detection moieties that include reporter moieties can also include optical label binding regions that are not part of reporter moieties, but instead are used to perform standardization or calibration functions. For example, detection moieties can include one or more verification regions, each of which generally includes an oligonucleotide sequence that can have some or all of the same properties as the label regions discussed previously.

The sequences of each of the verification regions are generally selected such that they are not complementary to any of the sequences of the optical labels. As such, during hybridization and detection of optical labels, none of the optical labels hybridizes to the verification regions, and no optical signals corresponding to optical labels hybridized to the verification regions are measured.

However, the procedure shown in FIG. 6 can be modified to include one or more standardization steps in which a standardization optical label, which generates an optical signal that is different from the optical signals generated by the optical labels that hybridize to the label regions of the reporter moieties, is introduced into the sample and hybridizes to one of the verification regions. The standardization optical label generates a standardization optical signal which is detected in the same manner discussed previously.

In general, detection moieties can include one or more (e.g., two or more, three or more, four or more, five or more, or even more) verification regions. Each of the verification regions in a detection moiety can be different (e.g., can have a different oligonucleotide sequences), or alternatively, one or more of the verification regions can be the same as another of the verification regions in a detection moiety.

Among a plurality of different types of detection moieties introduced into a sample, which bind to different RNAs in the sample, in some embodiments, each member of the plurality of detection moieties can include the same set of one or more verification regions. Alternatively, in certain embodiments, some or all of the verification regions among the plurality of detection moieties can differ. For example, where the detection moieties are part of probes that selectively bind to multiple different types of RNAs, each of the detection moieties can have a first verification region that is common to all detection moieties, and a second verification region that differs among some of the detection moieties. The sequence of the second verification region can depend, for example, on a particular class or grouping into which the RNA that is selectively bound falls. As one example, the sequence of the second verification region can be selected based on the origin of the RNA that is bound.

In this manner, measured optical signals corresponding to particular groups or classes of RNAs can be reliably distinguished from other optical signals in a multi-RNA assay by measuring signals corresponding to a standardization optical label that hybridizes to a standardization region of the detection moieties. Further, and in particular by measuring optical signals arising from a standardization optical label that hybridizes to a standardization region that is present in all detection moieties, optical signals measured in different portions of a sample can be corrected relative to one another. That is, the optical signal arising from the standardization optical label functions as a common reference optical signal across the sample, in which variations in the signal can be presumed to arise from sample inhomogeneity, variance in detection sensitivity, and other factors that may yield variations in imaging across a spatial field of view. Measured optical signals arising from optical labels that hybridize to the label regions of reporter moieties 106 can be corrected for such variations, using the optical signal from the standardization optical label as a reference signal. A wide variety of such corrections are possible, including but not limited to dividing optical signals arising from reporter moieties by the corresponding standardization optical label signal (i.e., a spatially-resolved correction), normalizing or scaling optical signals arising from reporter moieties to yield signals that deviate reproducibly from the reference standardization optical signal, and using the standardization optical signal to establish thresholds for positive and negative signal detection among the measured optical signals arising from optical labels that hybridize to label regions of the reporter moieties.

### Error Correction

In the methods discussed above, no restrictions have been applied to the selection of label regions in the reporter moieties of detection moieties that are part of probes for different RNAs in the sample. In some embodiments, various criteria and/or constraints can be applied to the distribution of the label regions, and error correction methods that are based on these criteria and/or constrains can be used to detect errors in measured optical signals in the sample that arise (or are presumed to arise) from optical labels that hybridize to the reporter moieties of the detection moieties, and to identify the absence of expected optical signals that are not measured in the sample. Certain types of errors can also be corrected to allow particular reporter moieties to be detected even when measured optical signals are in error. Examples of such criteria and constraints for the selection of label regions, and methods, compositions, and kits for performing error correction, are described in U.S. patent application Ser. No. 17/714,628, the entire contents of which are incorporated herein by reference.

### Compositions and Kits

The probes, detection moieties, reporter moieties, and optical labels described herein can be included in a variety of compositions. In some embodiments, the compositions can be used for analysis of a sample as described above in connection with FIGS. 6 and 7A-7D. Alternatively, or in addition, the compositions can be used for analysis of a sample according to different sets of steps and/or procedures.

In some embodiments, a composition—to which a sample can optionally be exposed—can include a plurality of probes. Each of the probes can include an optional capture moiety 102, and a detection moiety 104. The detection moiety 104 can include one or more reporter moieties 106. Reporter moieties 106 can have any of the label regions, and any number of label regions, described herein. The probes can also optionally include one or more verification regions, and any of the other features described herein.

In certain embodiments, the compositions can include any one or more of the species (i.e., probes, oligonucleotides, optical labels, and other species) described herein that are used to label the sample with probes. For example, the compositions can include any of the types of probes and other species described in connection with FIGS. 4 and 5.

Label regions of the reporter moieties in a composition can be selected such that, when the compositions are used to label samples, different types of RNA in the sample are labeled with different types of probes. Each type of probe can selectively label a different type of RNA in the sample. Typically, probes of the same type each include the same type of reporter moieties within the detection moieties of the probes, or optionally, reporter moieties that are indistinguishable variants of one another (i.e., reporter moieties in which the ordering of the label regions varies, but the label regions correspond to the same oligonucleotide sequences). Probes of each type have reporter moieties that differ from probes of other types of reporter moieties, without regard to the relative ordering of the label regions in each type of probe.

Compositions can generally include probes, species, and other components that are used to label a sample as described herein such that any number of different types of RNAs in the sample can be distinguishably labeled. In some embodiments, for example, compositions can be used to label 2 or more (e.g., 3 or more, 4 or more, 5 or more, 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 10000 or more, or even more) different types of RNA analytes in the sample, and can contain distinguishable probes, oligonucleotides, and other components of the same number.

Each population or group of probes, oligonucleotides, or another component within a composition can generally include any number of probes, oligonucleotides, or other component of a particular type. For example, the number of probes, oligonucleotides, or another component of a particular type can be 1 or more (e.g., 2 or more, 3 or more, 5 or more, 10 or more, 50 or more, 100 or more, 200 or more, 300 or more, 500 or more, 1000 or more, 2000 or more, 3000 or more, 5000 or more, 7000 or more, 10000 or more, 30000 or more, 50000 or more, or even more).

In some embodiments, compositions can include any of the reporter moieties described herein. The reporter moieties can be present as part of larger species, i.e., as part of probes or oligonucleotides, such as probes and oligonucleotides that are used as templates for RCA-based extension of other oligonucleotides, or alternatively, can be uncoupled to other moieties and entities within the composition. The compositions can also include reporter moieties that are present as part of detection moieties, with the detection moieties either being present as part of probes, or alternatively, uncoupled within the composition.

Label regions of the reporter moieties in such circumstances can be selected to yield different types of reporter moieties in the composition, and optionally, different types of detection moieties in the composition, and different types of probes in the composition. In particular, the composition can include groups or populations of different types of reporter moieties, where reporter moieties of the same type are the same or differ from one another merely in the relative ordering of their label regions, and reporter moieties of each type differ from reporter moieties of different types within the composition.

Compositions can generally include populations or groups of different types of reporter moieties. In some embodiments, for example, compositions can include populations or groups of 2 or more (e.g., 3 or more, 4 or more, 5 or more, 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 10000 or more, or even more) different types of reporter moieties.

Each population or group of a different type of reporter moiety within a composition can generally include any number of reporter moieties. For example, the number of reporter moieties of a particular type can be 1 or more (e.g., 2 or more, 3 or more, 5 or more, 10 or more, 50 or more, 100 or more, 200 or more, 300 or more, 500 or more, 1000 or more, 2000 or more, 3000 or more, 5000 or more, 7000 or more, 10000 or more, 30000 or more, 50000 or more, or even more).

In general, compositions can include populations or groups of different types of detection moieties, each of which includes one or more reporter moieties of the same (or indistinguishable) type. In some embodiments, for example, compositions can include populations or groups of 2 or more (e.g., 3 or more, 4 or more, 5 or more, 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 10000 or more, or even more) different types of detection moieties.

Each population or group of a different type of detection moiety within a composition can generally include any number of detection moieties. For example, the number of detection moieties of a particular type can be 1 or more (e.g., 2 or more, 3 or more, 5 or more, 10 or more, 50 or more, 100 or more, 200 or more, 300 or more, 500 or more, 1000 or more, 2000 or more, 3000 or more, 5000 or more, 7000 or more, 10000 or more, 30000 or more, 50000 or more, or even more).

In some embodiments, compositions can include one or more additional components. For example, in some embodiments, compositions can include one or more buffer solutions. Examples of suitable buffer solutions include, but are not limited to, saline-sodium citrate (SSC), phosphate-buffered saline (PBS), and tris-ethylenediaminetetraacetic acid (tris-EDTA).

In certain embodiments, compositions include one or more optical labels. The optical labels can include any of the optical labels described herein. Typically, as described in connection with FIGS. 6 and 7A-7D, a sample is exposed to optical labels in groups or pools, where each such group includes 2 or more (e.g., 3 or more, 4 or more, 5 or more, 6 or more, 8 or more, or even more) different optical labels. Within a composition, the optical labels can optionally be divided into sub-compositions, each of which contains a group or pool of optical labels. In certain embodiments, among sub-compositions, each optical label can be present in only one sub-composition. Alternatively, in some embodiments, one or more optical labels can be present in more than one sub-composition.

The sample is exposed to a composition that includes optical labels to hybridize some or all of the optical labels to complementary label regions of reporter moieties, as described above. In certain embodiments, the sample is exposed to the entire composition of optical labels at the same time. Alternatively, in some embodiments, the sample is exposed to sub-compositions of the composition sequentially as discussed previously.

In some embodiments, optical labels are present in the same composition as the probes, oligonucleotides, and/or reporter moieties discussed above. In certain embodiments, optical labels are present in a different composition to which the sample is exposed after exposure to one or more compositions that include(s) the probes, oligonucleotides, and/or reporter moieties.

Compositions that include optical labels can also optionally include additional components. For example, in some embodiments, compositions can include one or more buffer solutions. Examples of suitable buffer solutions include, but are not limited to, saline-sodium citrate (SSC), phosphate-buffered saline (PBS), and tris-ethylenediaminetetraacetic acid (tris-EDTA).

One or more of the compositions described above can be included as part of a kit for analyzing a sample. The kit can include a housing or packaging that encloses the contents of the kit. Compositions can be contained within containers in the kit; such containers can be formed from a wide variety of materials, including (but not limited to) plastics and glass.

Kits can optionally include a variety of other components as well. For example, in some embodiments, kits can include one or more dehybridization reagents. Examples of such reagents include, but are not limited to, sodium hydroxide, dimethyl sulfoxide (DMSO), formamide, SDS, methanol, and ethanol.

In certain embodiments, kits can include one or more buffer solutions. Examples of suitable buffer solutions include, but are not limited to, saline-sodium citrate (SSC), phosphate-buffered saline (PBS), and tris-ethylenediaminetetraacetic acid (tris-EDTA).

Kits can also optionally include instructions printed or otherwise recorded on any of a variety of different media (e.g., paper, computer readable storage media) that describe the use of certain components of the kits in assays targeting RNAs of various types in samples.

### Sample Analysis Systems and Components

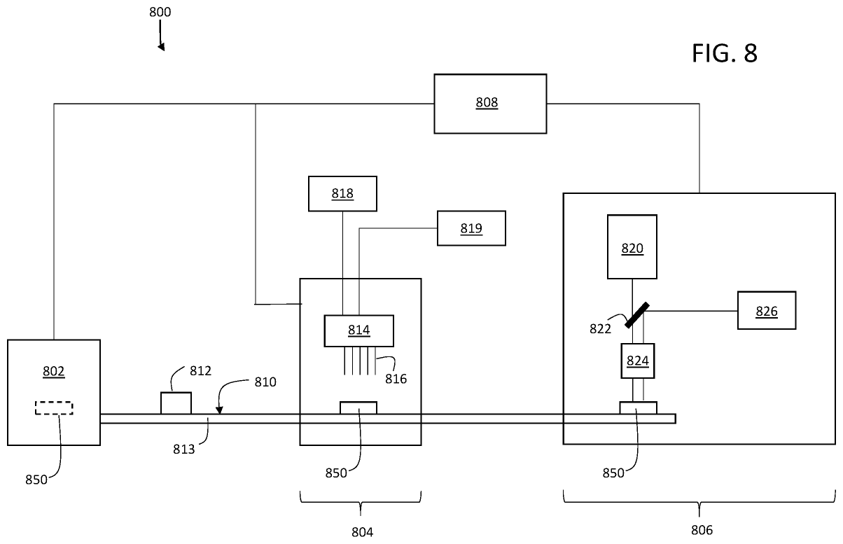

The methods described herein can be implemented in a variety of different analysis systems, including systems that perform some or all of the steps in semi-automated or fully automated fashion. One example of such a system 800 is shown schematically in FIG. 8. System 800 includes a storage unit 802, a labeling station 804, an imaging station 806, and a translation apparatus 810. Each of these components is connected to controller 808, which includes one or more electronic processors that perform control functions associated with any of the steps and/or analysis functions described herein.

Translation apparatus 810 includes a slide handler 812 that attaches to individual slides on which biological samples are disposed for analysis, and transport the slides between different locations in the system. An example of a slide on which a sample is disposed is indicated as slide 850 in FIG. 8. Slide handler 812 can be implemented in a number of ways. In some embodiments, for example, slide handler 812 is a grasper and includes one or more arms or fingers that exert pressure on surfaces of slide 850 to lift and transport slide 850. In certain embodiments, slide handler 812 includes a member with one or more suction ports that uses reduced pressure to lift individual slides 850. In certain embodiments, slide handler 812 includes one or more members that are inserted under slides 850 to lift the slides. In general, slide handler 812 can permit both rotational displacements of slides 850 about three orthogonal axes, and translations along three orthogonal axes.

Translation apparatus 810 can also include a track or conveyor 813 that carries individual slides 850 or containers of slides between locations in system 800. In some embodiments, track 813 is a linear track that moves back and forth along a single direction between locations. In certain embodiments, track 813 is a continuous track (e.g., circular, elliptical, or another continuous shape) that circulates among locations in the system.

During operation, controller 808 can transmit appropriate control signals to translation apparatus 810 to retrieve one or more slides 850 from storage unit 802, and to deposit one or more slides into storage unit 802, as discussed above. Further, controller 808 can transmit control signals to translation apparatus 810 to activate labeling station 804 to deliver fluids, reagents, and compositions to slide 850, and remove fluids, reagents, compositions (and components thereof) from slide 850. Labeling station 804 includes a fluidic apparatus 814 connected to one or more reservoirs 818 and to one or more pumps and/or vacuum sources 819. The fluidic apparatus 814 includes one or more fluid conduits (e.g., syringes, tubes, pipettes) are coupled to the one or more reservoirs 818, and are optionally positionable relative to slide 850, to deliver any of the reagents and/or compositions disclosed herein to the biological sample disposed on slide 850. Fluid conduits that are optionally positionable can be formed from flexible conduit materials such as any one or more of various conventional polymer materials, and coupled to robotic actuators (not shown in FIG. 8) that are connected to, and receive positioning instructions from, controller 808.

During operation of the system, controller 808 transmits signals to translation apparatus 810 to position slide 850 within labeling station 804, and transmits signals to the fluidic apparatus 814 to cause one or more of the fluid conduits to deliver fluids, reagents, and/or compositions from reservoirs 818 to the biological sample disposed on slide 850. Further controller 808 transmits signals to activate the one or more pumps and/or vacuum sources 819 to selectively remove fluids, reagents, and/or compositions (and components thereof) from the biological sample. In this manner, controller 808 (transmitting instructions to labeling station 804) can implement any of the steps described herein in automated fashion.

Imaging station 806 includes a radiation source 820, an objective lens 824, a beam splitter 822, and an image detector 826. Radiation source 820 can include any one or more of a variety of different sources, including but not limited to LEDs, laser diodes, metal halide sources, incandescent sources, and fluorescent sources. Image detector 826 can include one or more different detector types, including but not limited to CCD detectors and CMOS detectors. During operation of system 800, to obtain an image of a biological sample disposed on slide 850, controller 808 activates translation apparatus 810 to position slide 850 within imaging station 806. Controller 808 then transmits control signals to the components of the imaging station, activating source 820 to deliver illumination radiation to the sample which passes through beam splitter 822 and objective lens 824 and is incident on the sample. Emitted light from the sample passes through objective lens 824, is reflected from beam splitter 822, and is incident on image detector 826, which measures an image of the emitted light.

In FIG. 8, the imaging station is configured to obtain a fluorescence or reflected-light image of the sample. However, it should be appreciated that in some embodiments, detector 826 can be positioned on an opposite side of slide 850 from source 820 to measure a transmitted-light image of the sample. In certain embodiments, imaging station 806 includes multiple detectors for measuring both transmitted- and reflected- or emitted-light (e.g., fluorescence) images of the sample. Under the control of controller 808, imaging station 806 can obtain any of the different types of images corresponding to any of the different types of dyes, stains, probes, and labeling agents described herein.

Further, it should be noted that while in the example system 800 of FIG. 8 the labeling station 804 and imaging station 806 are separate, in some embodiments the labeling and imaging stations can be combined into a single station under the control of controller 808, and which performs both sample labeling and imaging functions as described herein.

Each of the steps in sample analysis consumes a certain amount of time, and improved efficiency can be obtained by translating multiple slides 850 among multiple locations in system 800, and performing certain operations in parallel. For example, during analysis of multiple slides 850, a first slide on which a first sample is disposed can be positioned at the labeling station, and the fluidic apparatus can deliver one or more probes to the sample and incubate the sample with the delivered probes. At the same time, a second slide on which a second sample is disposed can be positioned at the imaging station to obtain one or more images of the sample. A third slide on which a third sample is disposed can be positioned at the labeling station, where controller 808 activates the one or more pumps and/or vacuum sources 819 to remove fluids, reagents, and/or compositions (and components thereof) from the sample disposed on the third slide. A fourth slide on which a fourth sample is disposed can be positioned at the labeling station, where controller 808 activates the fluidic apparatus 814 to deliver one or more fluids, reagents, and/or compositions, and incubates the fourth sample with the fluids, reagents, and/or compositions. Other slides 850 can also be processed—undergoing any of the operations discussed herein—at the same time.

As each slide reaches the end of a set of one or more steps in a preparation or analysis workflow, the slide is transported by translation apparatus 810 to a different location in the system—to a different station, or to a different location within the same station, for example. In this manner, system 800 can analyze multiple samples in parallel, ensuring that the duty cycles of the components of system 800 remain relatively high, increasing overall throughput of the system for multiple samples relative to simple linear processing of individual samples.

Additional aspects and features of system 800 are described in U.S. Patent Application Publication No. US 2020/0393343, the entire contents of which are incorporated herein by reference.

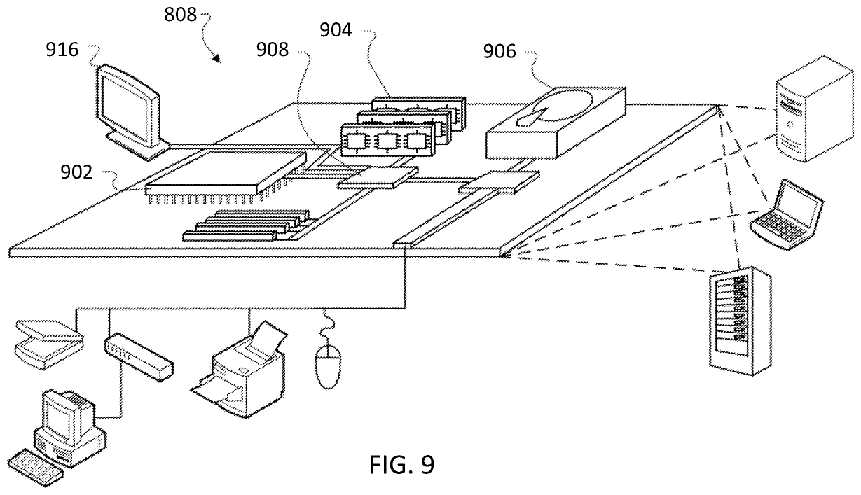

FIG. 9 shows an example of controller 808, which may be used with the systems and methods disclosed herein. Controller 808 can include one or more processors 902, memory 904, a storage device 906 and interfaces 908 for interconnection. The processor(s) 902 can process instructions for execution within the controller, including instructions stored in the memory 904 or on the storage device 906. For example, the instructions can instruct the processor 902 to perform any of the analysis and control steps disclosed herein.

The memory 904 can store executable instructions for processor 902, information about parameters of the system such as excitation and detection wavelengths, and measured image information. The storage device 906 can be a computer-readable medium, such as a floppy disk device, a hard disk device, an optical disk device, or a tape device, a flash memory or other similar solid state memory device, or an array of devices, including devices in a storage area network or other configurations. The storage device 906 can store instructions that can be executed by processor 902 as described above, and any of the other information that can be stored by memory 804.

In some embodiments, controller 808 can include a graphics processing unit to display graphical information (e.g., using a GUI or text interface) on an external input/output device, such as display 916. The graphical information can be displayed by a display device (e.g., a CRT (cathode ray tube) or LCD (liquid crystal display) monitor) for displaying any of the information, such as measured and calculated spectra and images, disclosed herein. A user can use input devices (e.g., keyboard, pointing device, touch screen, speech recognition device) to provide input to controller 808. In some embodiments, one or more such devices can be part of controller 808.

A user of system 800 can provide a variety of different types of instructions and information to controller 808 via input devices. The instructions and information can include, for example, information about any of the parameters (e.g., dyes, labels, probes, reagents, conditions) associated with any of the workflows described herein, calibration information for quantitative analysis of sample images, and instructions following manual analysis of sample images by a technician. Controller 808 can use any of these various types of information to perform the methods and functions described herein. It should also be noted that any of these types of information can be stored (e.g., in storage device 906) and recalled when needed by controller 808.

The methods disclosed herein can be implemented by controller 808 by executing instructions in one or more computer programs that are executable and/or interpretable by the controller 808. These computer programs (also known as programs, software, software applications or code) include machine instructions for a programmable processor, and can be implemented in a high-level procedural and/or object-oriented programming language, and/or in assembly/machine language. For example, computer programs can contain the instructions that can be stored in memory 904, in storage unit 906, and/or on a tangible, computer-readable medium, and executed by processor 902 as described above. As used herein, the term “computer-readable medium” refers to any computer program product, apparatus and/or device (e.g., magnetic discs, optical disks, memory, Programmable Logic Devices (PLDs), ASICs, and electronic circuitry) used to provide machine instructions and/or data to a programmable processor, including a machine-readable medium that receives machine instructions.

By executing instructions as described above (which can optionally be part of controller 808), the controller can be configured to implement any one or more of the various steps described in connection with any of the workflows herein. For example, controller 808 can selectively direct particular fluids, reagents, and/or compositions to particular regions of a biological sample (e.g., by activating fluidic apparatus 814), and selectively remove fluids, reagents, and/or compositions (and components thereof) from the sample. Controller 808 can select particular probes (with particular reporter moieties) to deliver to the biological sample, and select particular optical labels (including combinations of optical labels, according to the criteria described above) for delivery to the sample. Controller 808 can obtain measurements of optical signals from optical labels in the sample, and determine locations of the optical signals based on images received from the imaging station.

Controller 808 can determine the locations of particular analytes in the sample based on the locations of the optical signals and the types of optical labels delivered to the sample in one or more cycles of label exposure and imaging. For example, during each cycle of the workflow (see FIG. 6, for example), controller 808 can identify particular label regions present at particular locations in the sample. Based on combinations of label regions present at particular locations in the sample, controller 808 (e.g., by comparing to stored information about combinations of label regions in specific types of probes) can identify particular analytes at locations in the sample. Controller 808 can also implement error correction steps as described herein in circumstances where measured signals from optical labels cannot be unambiguously used to identify particular analytes at locations in the sample.

### Applications

The hybridization and signal detection methods, compositions, and kits described herein can be used to detect many different types of RNAs in a wide variety of different samples. The methods, compositions, and kits do not depend on the manner in which RNAs in the sample bind to probes, nor do they depend on the nature of the RNAs. Further, other than the aspects of the label regions described herein, probes can have a wide variety of different structural compositions and functionalities. Provided they do not interfere with the hybridization of optical labels, additional structural features of the probes can be present without interfering with the methods described herein.

Samples to which the methods, compositions, and kits can be applied include tissue samples (e.g., tissue sections), including fresh, fresh-frozen, and formalin-fixed, paraffin-embedded (FFPE) samples. Other samples include, but are not limited to, cell and tissue lysates, body fluids (e.g., blood, urine, saliva, lymph fluid, renal fluid, and other such fluids), thick tissue (e.g., solid tumor tissue, biopsy samples), individual cells, suspensions of cells and other biological materials, and aspirated samples.

Additional applications and aspects of the methods and systems disclosed herein are described, for example, in U.S. patent application Ser. No. 17/714,628, the entire contents of which are incorporated herein by reference.

### Other Embodiments

While this disclosure describes specific implementations, these should not be construed as limitations on the scope of the disclosure, but rather as descriptions of features in certain embodiments. Features that are described in the context of separate embodiments can also generally be implemented in combination in a single embodiment. Conversely, various features that are described in the context of a single embodiment can also be implemented in multiple embodiments separately or in any suitable sub-combination. Moreover, although features may be described above as present in certain combinations and even initially claimed as such, one or more features from a claimed combination can generally be excised from the combination, and the claimed combination may be directed to a sub-combination or variation of a sub-combination.

In addition to the embodiments expressly disclosed herein, it will be understood that various modifications to the embodiments described may be made without departing from the spirit and scope of the disclosure. Accordingly, other embodiments are within the scope of the following claims.

What is claimed is:

1. A method, comprising:
contacting a biological sample with a first probe, wherein the first probe comprises a capture moiety having an oligonucleotide sequence that selectively binds to a RNA in the sample, and a secondary oligonucleotide region that does not bind to the RNA;
contacting the sample with a second probe, wherein the second probe comprises a probe binding region that is complementary to, and hybridizes to, a portion of the secondary oligonucleotide region, and comprises a reporter moiety; and
extending the secondary oligonucleotide region using the second probe as a template to generate an extended secondary oligonucleotide region comprising multiple copies of the reporter moiety,
wherein the reporter moiety comprises a plurality of label regions each comprising an oligonucleotide sequence; and
wherein one or more of the label regions of the reporter moiety are different from the other label regions of the reporter moiety.

2. The method of claim 1, wherein:
the probe binding region is a first probe binding region that is complementary to, and hybridizes to, a first portion of the secondary oligonucleotide region;
the second probe comprises a second probe binding region that is complementary to, and hybridizes to, a second portion of the secondary oligonucleotide region different from the first portion of the secondary oligonucleotide region.

3. The method of claim 2, further comprising, prior to extending the secondary oligonucleotide region, joining the first and second probe binding regions.

4. The method of claim 3, comprising performing a ligation reaction to join the first and second probe binding regions.

5. The method of claim 1, wherein the second probe comprises a circular nucleic acid.

6. The method of claim 1, wherein extending the secondary oligonucleotide region comprises performing a rolling circle amplification reaction to extend the secondary oligonucleotide region.

7. The method of claim 1, wherein the extended secondary oligonucleotide region comprises at least 10 copies of the reporter moiety.

8. The method of claim 7, wherein the extended secondary oligonucleotide region comprises at least 50 copies of the reporter moiety.

9. The method of claim 8, wherein the extended secondary oligonucleotide region comprises at least 100 copies of the reporter moiety.

10. The method of claim 1, wherein the reporter moiety comprises at least 3 label regions.

11. The method of claim 10, wherein the reporter moiety comprises at least 4 label regions.

12. The method of claim 1, wherein one of the label regions of the reporter moiety is different from the other label regions of the reporter moiety.

13. The method of claim 12, wherein two of the label regions of the reporter moiety are different from the other label regions of the reporter moiety.

14. The method of claim 1, wherein the reporter moiety comprises at least two different types of label regions, wherein each different type of label region comprises a unique oligonucleotide sequence.

15. The method of claim 14, wherein the reporter moiety comprises at least three different types of label regions.

16. The method of claim 15, wherein the reporter moiety comprises at least four different types of label regions.

17. The method of claim 14, wherein each of the label regions in the reporter moiety is a different type of label region.

18. The method of claim 1, wherein each label region comprises at least 15 nucleotides.

19. The method of claim 18, wherein each label region comprises at least 30 nucleotides.

20. The method of claim 1, wherein each label region comprises a same number of nucleotides.

21. The method of claim 1, further comprising:
(a) exposing the sample to a plurality of optical labels, wherein each of the optical labels comprises an oligonucleotide having a sequence, and a species that generates an optical signal;
(b) measuring optical signals generated by optical labels of the plurality of optical labels that hybridize to complementary label regions of the reporter moieties;
(c) repeating steps (a) and (b) with different pluralities of optical labels;
(d) identifying one or more of the reporter moieties in the sample based on the measured optical signals; and
(e) determining one or more locations of the RNA in the sample based on the one or more identified reporter moieties.

22. The method of claim 21, wherein the species that generates the optical signal is a fluorescent moiety.

23. The method of claim 21, wherein the species that generates the optical signal comprises at least one fluorescent nucleotide.

24. The method of claim 21, wherein measuring optical signals generated by the optical labels comprises obtaining at least one image of the optical labels in the sample, and identifying optical signals corresponding to the optical labels in the at least one image.

25. The method of claim 21, wherein each plurality of optical labels in step (a) comprises a same number of different types of optical labels.

26. The method of claim 21, wherein each plurality of optical labels in step (a) comprises 3 or more different types of optical labels.

27. The method of claim 26, wherein each plurality of optical labels in step (a) comprises 5 or more different types of optical labels.

28. The method of claim 21, further comprising repeating step (a) until the sample has been exposed to a set of optical labels, wherein each label region of the reporter moieties has a complementary optical label in the set of optical labels.

29. The method of claim 21, further comprising exposing the sample to at least one of the plurality of optical labels more than once.

30. The method of claim 21, wherein each time step (a) is performed, each member of the plurality of optical labels in step (a) comprises a species that generates a different optical signal.

31. The method of claim 30, wherein the different optical signals have different spectral distributions.

32. The method of claim 21, wherein among the plurality of optical labels:
at least two of the optical labels comprise a common species that generates the optical signal; and
the at least two of the optical labels are exposed to the sample during different repetitions of step (a).

33. The method of claim 21, wherein among the plurality of optical labels:
first and second optical labels each comprise a first species that generates the optical signals of the first and second optical labels;
third and fourth optical labels each comprise a second species that generates the optical signals of the third and fourth optical labels; and
the first and second species are different.

34. The method of claim 33, wherein the optical signals generated by the first and second species are different.

35. The method of claim 34, further comprising:
exposing the sample to the first and second optical labels during different repetitions of step (a); and
exposing the sample to the third and fourth optical labels during different repetitions of step (a).

36. The method of claim 21, further comprising, for at least one of the optical labels, removing the at least one of the optical labels from the sample after measuring an optical signal generated by the at least one of the optical labels.

37. The method of claim 36, wherein removing the at least one of the optical labels comprises dehybridizing the at least one of the optical labels from one or more label regions of the reporter moieties.

38. A method, comprising:
contacting a biological sample with a first probe and a second probe, wherein the first probe comprises a first capture moiety having an oligonucleotide sequence that selectively binds to a first portion of a RNA in the sample and a secondary oligonucleotide region that does not bind to the RNA, and wherein the second probe comprises a second capture moiety having an oligonucleotide sequence that selectively binds to a second portion of the RNA and a secondary oligonucleotide region that does not bind to the RNA;
contacting the sample with a third probe comprising a first probe binding region that is complementary to, and hybridizes to, a portion of the secondary oligonucleotide region of the first probe, and a second probe binding region that is complementary to, and hybridizes to, a portion of the secondary oligonucleotide region of the second probe;
contacting the sample with a fourth probe comprising:
a third probe binding region that is complementary to, and hybridizes to, a portion of the secondary oligonucleotide region of the first probe;
a fourth probe binding region that is complementary to, and hybridizes to, a portion of the secondary oligonucleotide region of the second probe; and
a reporter moiety comprising a plurality of label regions each comprising an oligonucleotide sequence; and
extending the secondary oligonucleotide region of the first probe using the fourth probe as a template to generate an extended secondary oligonucleotide region comprising multiple copies of the reporter moiety,
wherein one or more of the label regions of the reporter moiety are different from the other label regions of the reporter moiety.

39. The method of claim 38, further comprising, prior to extending the secondary oligonucleotide region of the first probe, joining the third and fourth probes to form a circularized probe comprising the reporter moiety.

40. The method of claim 39, comprising performing a ligation reaction to join the third and fourth probes.

41. The method of claim 38, wherein extending the secondary oligonucleotide region of the first probe comprises performing a rolling circle amplification reaction to extend the secondary oligonucleotide region.

42. The method of claim 38, wherein the extended secondary oligonucleotide region of the first probe comprises at least 10 copies of the reporter moiety.

43. The method of claim 42, wherein the extended secondary oligonucleotide region of the first probe comprises at least 50 copies of the reporter moiety.

44. The method of claim 43, wherein the extended secondary oligonucleotide region of the first probe comprises at least 100 copies of the reporter moiety.

45. The method of claim 38, wherein the reporter moiety comprises at least 3 label regions.

46. The method of claim 45, wherein the reporter moiety comprises at least 4 label regions.

47. The method of claim 38, wherein one of the label regions of the reporter moiety is different from the other label regions of the reporter moiety.

48. The method of claim 47, wherein two of the label regions of the reporter moiety are different from the other label regions of the reporter moiety.

49. The method of claim 38, wherein the reporter moiety comprises at least two different types of label regions, wherein each different type of label region comprises a unique oligonucleotide sequence.

50. The method of claim 49, wherein the reporter moiety comprises at least three different types of label regions.

51. The method of claim 50, wherein the reporter moiety comprises at least four different types of label regions.

52. The method of claim 49, wherein each of the label regions in the reporter moiety is a different type of label region.

53. The method of claim 38, wherein each label region comprises at least 15 nucleotides.

54. The method of claim 53, wherein each label region comprises at least 30 nucleotides.

55. The method of claim 38, wherein each label region comprises a same number of nucleotides.

56. The method of claim 38, further comprising:
(a) exposing the sample to a plurality of optical labels, wherein each of the optical labels comprises an oligonucleotide having a sequence, and a species that generates an optical signal;
(b) measuring optical signals generated by optical labels of the plurality of optical labels that hybridize to complementary label regions of the reporter moieties;
(c) repeating steps (a) and (b) with different pluralities of optical labels;
(d) identifying one or more of the reporter moieties in the sample based on the measured optical signals; and
(e) determining one or more locations of the RNA in the sample based on the one or more identified reporter moieties.

57. The method of claim 56, wherein the species that generates the optical signal is a fluorescent moiety.

58. The method of claim 56, wherein the species that generates the optical signal comprises at least one fluorescent nucleotide.

59. The method of claim 56, wherein measuring optical signals generated by the optical labels comprises obtaining at least one image of the optical labels in the sample, and identifying optical signals corresponding to the optical labels in the at least one image.

60. The method of claim 56, wherein each plurality of optical labels in step (a) comprises a same number of different types of optical labels.

61. The method of claim 56, wherein each plurality of optical labels in step (a) comprises 3 or more different types of optical labels.

62. The method of claim 61, wherein each plurality of optical labels in step (a) comprises 5 or more different types of optical labels.

63. The method of claim 56, further comprising repeating step (a) until the sample has been exposed to a set of optical labels, wherein each label region of the reporter moieties has a complementary optical label in the set of optical labels.

64. The method of claim 56, further comprising exposing the sample to at least one of the plurality of optical labels more than once.

65. The method of claim 56, wherein each time step (a) is performed, each member of the plurality of optical labels in step (a) comprises a species that generates a different optical signal.

66. The method of claim 65, wherein the different optical signals have different spectral distributions.

67. The method of claim 56, wherein among the plurality of optical labels:
at least two of the optical labels comprise a common species that generates the optical signal; and
the at least two of the optical labels are exposed to the sample during different repetitions of step (a).

68. The method of claim 56, wherein among the plurality of optical labels:
first and second optical labels each comprise a first species that generates the optical signals of the first and second optical labels;
third and fourth optical labels each comprise a second species that generates the optical signals of the third and fourth optical labels; and
the first and second species are different.

69. The method of claim 68, wherein the optical signals generated by the first and second species are different.

70. The method of claim 69, further comprising:
exposing the sample to the first and second optical labels during different repetitions of step (a); and
exposing the sample to the third and fourth optical labels during different repetitions of step (a).

71. The method of claim 56, further comprising, for at least one of the optical labels, removing the at least one of the optical labels from the sample after measuring an optical signal generated by the at least one of the optical labels.

72. The method of claim 71, wherein removing the at least one of the optical labels comprises dehybridizing the at least one of the optical labels from one or more label regions of the reporter moieties.
